SEQUENCE LISTING (70 sequences)

SEQ ID NO: 1 - DNA, 357 bp, Homo Sapiens
aatacagattacctcttctgtctttaaagtttcagcttatgattctgctggtatgtgctatccttcagcaaagaatcatgaacagacattttcttactttattgtggatcctatcaggcgtcaccttcatgttttataccactgttatggtgtgggagacatgtcttaatgttctttcagattatgtacctctactattttgtatttatcatttttctatcttaatactaacttatagataaacatatactttgcaaattaattcaagaaaaatgtaaggagcctacttagagcagaagaaagcaaacaccaagatgcctttttaaaaatttgtgtggaatactaaccggggatcaa

SEQ ID NO: 2 - DNA, 365 bp, Homo Sapiens
  misc_feature (30)..(30): n is a, c, g, or t
cttccagtctctgagctctaaggagatcancagccggctccgccagtggtccatgctgggcagaatcaaggcgcaggcgttcggctttgaccagacctttcagtcctatcggaaggatgatttcgttatggcttttttcaaagacccaaatgttattcccaatttgaagttactttcagattcttctggacaatggatcatattaggttttgctagtggaagactcagaaaaatatgaaatattcagccaaccagatagagaagagttcctgttttgtcttttcaaacatctttgccttggtggagccctttgtcaatatgaggatgtgattagcccatatctggaaacaacaaagcttatctat

SEQ ID NO: 3 - DNA, 466 bp, Homo Sapiens
  misc_feature (435)..(435): n is a, c, g, or t
caacatgctgctcaacaacggcttcagggagtcggaccagcttttcaaacaatacagaaaaagttttttgacttctgagaaaacctcagcgcttcctggagaaactcaaaggcgcttgtaagagatgttgagtcagcaggcctctgagcctcttctctgcctaaaaccatgtcagattcttgggaggactggaaggacttgcaggctgtccatgatttctagacaagtcatctgctcacagacaagaatgtgtctctgccttgctcctggagacgataggagagtccacagatgcagagatgtaagaaaaggagaagaaatgaaaagcagaaatggtttaagggaactggcgggaaagacaggaaactcatctgttgactttttccatcctcttcaactcagtggtgtgttggaaaaaggcatcttttttccccnaactattacactgtctgaaaaggtcctaatt

SEQ ID NO: 4 - DNA, 287 bp, Homo Sapiens
gttccagtgctggaaaacgatattcacctcagtttgtaaaggctgccaagttactccattggaatggacatttgaagccatggggaaggactgcttcatatactgatgtttgggaaaaatggtatattccagacccaacaggcaaattcaacctaatccgaagatataccgagatctcaaacataaagtgaaacagaatttgaactgtaagcaagcatttctcaggaagtcctggaagatagcatgcgtgggaagtaacagttgctaggcttcaatgcctatcggta

SEQ ID NO: 5 - DNA, 523 bp, Homo Sapiens
gtttcatggacggtttcaccacacttaaggagaacataatgaggttctggagatccccgaaccccgggtcctgggtgtcctgcgaacaggtgctgctggcgctgctgctgctgctggcgctgctgctgccgctgctcagcgggggcctgcacctgctgctcaagtgaggccccggcggctcagggcggggctggccccacccccatgaccactgccctggaggtggcggcctgctgctgttatctttttaactgttttctcatgatgcctttttatatttaaaccccgagatagtgctggaacactgctgaggttttatactcaggttttttgttttttttttattccagttttcgttttttctaaaagatgaattcctatggctctgcaattgtcaccggttaactgtggcctgtgtttaggaagagccattcactcctgccctgccacacggcaggtagcagggggagtgctggtacgcccctgtgtgatatgttgatccctcggcaaagaatctactgga

SEQ ID NO: 6 - DNA, 546 bp, Homo Sapiens
agtggcatgaaggactctactgtggtgtggccccctctgcaaagtgcatttggagaccaggttccatgccaacaaactatgagctgtactatggcttcacaaggtttgctattgagctcaatgagttagatccagtactaaaagatctccttccaccaacagacgcccggttccggccagatcaaagatttttggaagaaggaaatttagaagctgcagcatcagagaagcaaagagtagaggaactccagagatctcggagacgatatatggaagaaaacaatcttgaacatataccaaaattttttaaaaaagttattgatgccaatcaaagagaagcctgggtttctaacgacacctactgggagcttcgaaaggaccctgggtttagcaaagtagacagccctgttctttggtagactgggaatgtagagctagccaacatatcacattctgaatgaataaataactatgcacaattatgtttcttatagctatgtgtggtttctgggtcaactgaaaacctaccatttgcttttctattca

SEQ ID NO: 7 - DNA, 372 bp, Homo Sapiens
  misc_feature (137)..(137): n is a, c, g, or t
ttcctgtgcttacgtattttcagaaattttagttggtgtgcataccacaatttgtattttatacttaaatatacatttcatggttgtatatttatttgatcaagttcttttatatcaaataacagagcttggcattnggttgcttgctcataactcggcagtaagcatttttaagcatgaagtnattctnnngttgaattttattcttgatatggggcttaggagtgagatttctggtctacagaaatgatcatgntcatgaattttgacatttatcgtcatgatgctttgcacaagggctgnaccaatttagatcatctttagcggtgaatgagtggattaattttactacaactactgatggtggtggtg

SEQ ID NO: 8 - DNA, 456 bp, Homo Sapiens
  misc_feature (156)..(156): n is a, c, g, or t
tatgttgtcagtctacacttgtggaagcaaatatcttgtttaatcaagatgatgtctagtgtcacctaaataatgcaaaaagtttaattctggatgaattcagctttattcaagatccacatattcaagtatcattccacagatattcacagaacncagaaatatctgtgttctgccttatgcctgtggctaagggatgcaaagtagaattgctttacattgactatatatgtgacatgtacgttgctgttttttttaaaataactttatcatgatattcaggtagatcctgggttctagaatatttaaaacaaaaggataaaatgataaaccaaagagtcaacttgttaacttttcttttttaagagatgggttttcactagtatgcccagtctggactccaagtcctgggctcaaacgatcctccagcctcaggttcctgagtagctcaacatt

SEQ ID NO: 9 - DNA, 302 bp, Homo Sapiens
gtcttctcagtcctggaggatgactggaacttcctggaatccttttatttttgttttatttccctgagcaccattggcctgggggattatgtgcctggggaaggctacaatcaaaaattcagagagctctataagattgggatcacgtgttacctgctacttggccttattgccatgttggtagttctggaaaccttctgtgaactccatgagctgaaaaaattcagaaaaatgttctatgtgaagaaggacaaggacgaggatcaggtgcacatcatagagcatgaccaactgtccttctc

SEQ ID NO: 10 - DNA, 379 bp, Homo Sapiens
ggccctcgaggccaagaattcggcacgaggcgcgccttccaggtaaagcaaatgaaattgaaggactcgcgcatcaagctgatgagtgagatcctgaacggcatcaaggtgctgaagctgtacgcctgggagcccagcttcctgaagcaggtggagggcatcaggcagggtgagctccagctgctgcgcacggcggcctacctccacaccacaaccaccttcacctggatgtgcagccccttcctggtgaggcttggcacagggctgggtccctgcctccagggctctgggtgcccaggcatggccagggctcattggactctaccctgacaccacctccacgctgctcaggtgaccctgatcaccctctgggtgtacg

SEQ ID NO: 11 - DNA, 300 bp, Homo Sapiens
atggctactggggagctcggggacttgggtggctactacttcaggttcttgcctcagaaaaccttccagtctctgagctctaaggagatcaccagccggctccgccagtggtccatgctgggcagaatcaaggcgcaggcgttcggctttgaccagacctttcagtcctatcggaaggatgatttcgttatggcttttttcaaagacccaaatgttattcccaatttgaagttactttcagattcttctggacaatggatcatattaggttttgctagtggaagactcagaaaaatatga

SEQ ID NO: 12 - DNA, 1752 bp, Homo Sapiens
atggccggctcggagcagcagcggccgcggcggcgggacgacggagactcggacgcggcagcggcggcggcggcgcccctgcaggacgcggagctggccctggccggcatcaacatgctgctcaacaacggcttcagggagtcggaccagcttttcaaacaatacagaaatcatagcccactaatgagttttggagccagctttgtcagttttttgaatgccatgatgacatttgaggaagaaaaaatgcagttggcatgtgatgacttaaaaaccacagaaaaactgtgtgaaagtgaagaggctggagtaattgaaacaatcaagaataaaattaagaagaacgttgatgtccgaaaatccgccccctctatggttgatcggcttcagaggcagataatcatagctgactgccaggtttacctggctgtgctttcatttgtaaaacaagaattgtcagcttatatcaaaggtgggtggatccttaggaaagcctggaagatttacaataaatgctatctggacatcaatgcccttcaggagctgtatcagaagaagctaactgaagagtccttgacttctgatgctgcaaatgataatcacattgtggctgaaggggtgtctgaggagtctctgaacagactgaaaggtgctgttagctttggatatggcctttttcacctttgcatatccatggtgcccccaaacctgctcaaaatcatcaacctgctgggttttcctggagaccgcctacaggggctttcttcactgatgtatgcaagcgaaagtaaggacatgaaggcccctttagctacattagctctgctctggtatcatactgtagtccgcccgttttttgccttggatggcagtgataacaaggcaggcctggatgaagctaaggaaattctccttaaaaaagaagctgcttatccaaattcttccctctttatgtttttcaagggacggatacaacgactagagtgtcaaatcaacagtgccttgacatctttccacactgctttggaacttgcagtagaccagagagaaattcaacatgtctgtctgtatgaaattggttggtgcagcatgatagagctcaatttcaaggatgcatttgattcctttgagaggctaaaaaatgagtccaggtggtcccagtgctattatgcctacttgactgcagtttgtcagggagccactggtgatgtggatggggcacagattgtctttaaagaagttcagaaactcttcaaaaggaaaaacaatcagattgaacagttctcggtgaaaaaggcagagcgatttcggaagcaaaccccaaccaaagcgctctgtgtgttggcgtctattgaagtgttgtacttgtggaaagctcttccaaactgttccttccccaacctgcagaggatgagtcaagcttgccatgaagtggatgactcatctgttgttggattaaagtatttgcttcttggtgccatacacaaatgtctaggaaactcagaagatgctgttcagtacttccagcgagctgttaaagatgaattgtgtcgtcagaataatttatatgttcagccgtatgcctgttatgaacttggctgtcttctattagacaaaccagagactgtaggaagaggcagagctctacttcttcaagcaaaggaggatttctctggctacgactttgaaaacagattgcatgtccgcatccatgctgctctggcctctctgagggaattggttcctcagtga

SEQ ID NO: 13 - DNA, 1116 bp, Homo Sapiens
atgtcattccgtaaagtaaacatcatcatcttggtcctggctgttgctctcttcttactggttttgcaccataacttcctcagcttgagcagtttgttaaggaatgaggttacagattcaggaattgtagggcctcaacctatagactttgtcccaaatgctctccgacatgcagtagatgggagacaagaggagattcctgtggtcatcgctgcatctgaagacaggcttgggggggccattgcagctataaacagcattcagcacaacactcgctccaatgtgattttctacattgttactctcaacaatacagcagaccatctccggtcctggctcaacagtgattccctgaaaagcatcagatacaaaattgtcaattttgaccctaaacttttggaaggaaaagtaaaggaggatcctgaccagggggaatccatgaaacctttaacctttgcaaggttctacttgccaattctggttcccagcgcaaagaaggccatatacatggatgatgatgtaattgtgcaaggtgatattcttgccctttacaatacagcactgaagccaggacatgcagctgcattttcagaagattgtgattcagcctctactaaagttgtcatccgtggagcaggaaaccagtacaattacattggctatcttgactataaaaaggaaagaattcgtaagctttccatgaaagccagcacttgctcatttaatcctggagtttttgttgcaaacctgacggaatggaaacgacagaatataactaaccaactggaaaaatggatgaaactcaatgtagaagagggactgtatagcagaaccctggctggtagcatcacaacacctcctctgcttatcgtattttatcaacagcactctaccatcgatcctatgtggaatgtccgccaccttggttccagtgctggaaaacgatattcacctcagtttgtaaaggctgccaagttactccattggaatggacatttgaagccatggggaaggactgcttcatatactgatgtttgggaaaaatggtatattccagacccaacaggcaaattcaacctaatccgaagatataccgagatctcaaacataaagtga

SEQ ID NO: 14 - DNA, 483 bp, Homo Sapiens
atgtctgaagtaagacccctctccagagacatcttgatggagaccctcctgtatgagcagctcctggaacccccgaccatggaggttcttggcatgactgactctgaagaggacctggaccctatggaggacttcgattctttggaatgcatggagggcagtgacgcattggccctgcggctggcctgcatcggggacgagatggacgtgagcctcagggccccgcgcctggcccagctctccgaggtggccatgcacagcctgggtctggctttcatctacgaccagactgaggacatcagggatgttcttagaagtttcatggacggtttcaccacacttaaggagaacataatgaggttctggagatccccgaaccccgggtcctgggtgtcctgcgaacaggtgctgctggcgctgctgctgctgctggcgctgctgctgccgctgctcagcgggggcctgcacctgctgctcaagtga

SEQ ID NO: 15 - DNA, 2880 bp, Homo Sapiens
atgagttcagatgagaagggcatttcccctgctcataaaacatccactccaacccatagaagtgcctcctcttcaacatcctcccaaagggacagtaggcagagtattcacatactggagaggactgcttcctctagcaccgagccctctgtaagtcggcaattgctagaaccggagccagtccccctctccaaggaagctgacagctgggaaattatagaagggctgaaaataggccaaaccaatgtccagaaaccagacaaacatgagggctttatgctgaagaaaagaaaatggcctttaaaaggctggcacaagcgtttttttgtcctggataatggaatgttaaagtattcaaaggcaccactcgatattcagaaaggaaaggtccatgggagcatagatgtgggactctcagtcatgtcaattaaaaagaaagctcgaagaatagaccttgacaccgaagagcacatctatcatttgaaggtgaaatcccaggactggtttgatgcatgggtctccaaactgcgacatcatcggttgtatcgtcagaatgaaattgtgagatcaccaagagatgctagttttcacatatttccttcaacgtccacagctgaatcctcaccagctgctaatgtttctgtaatggatggaaagatgcaaccaaacagctttccgtggcagtcccctttaccatgcagcaatagcctccctgcaacgtgcacaactggccagagtaaagtggcagcctggttacaggactcggaagagatggacaggtgtgcagaagatcttgcacattgccagtcaaaccttgtggaacttagcaaactcctgcaaaatttggaaatacttcagagaactcagtcggcacctaactttactgacatgcaggctaactgtgtagatatttcaaagaaagacaagcgggtcacaagacgatggagaacaaaaagtgtcagcaaagatacaaaaatacaactgcaggaagggccacccgcgaagggccagttcagcacaactcggcgccggcagaggctagcggcagcagtggctacaacagttcctttcagtgctaccatgtcaccagttcgcttgcattcctccaaccccaacctttgtgcagatattgaatttcagactccccctagccacctcactgaccctctggaaagttcaacagattatacaaagctgcaagaagaattttgtctaatcgcacagaaagtgcattctcttttgaagtctgcatttaatagcatagctatagagaaggagaagctgaagcagatggtttccgagcaggatcacagtaaaggccacagcacgcagatggcacggctccgacagtcactgtctcaggcactcaaccagaatgctgaactaaggagtcggttgaacagaatacattcagagtctattatttgtgatcaggttgtcagtgtaaatattattcctagccctgatgaggctggtgagcaaatccatgtcagtctccccttatcacagcaagtagccaatgagagccgcctctccatgtcagagtctgtttctgagttctttgatgcccaagaggtgctcctctctgcaagttcgtcagagaatgaggcttcagatgatgagtcttacatcagtgatgtgagtgataatatatctgaagacaacaccagtgttgcagacaatatttctcggcaaatcctgaatggggagcttacaggaggggccttccgaaatgggcgtcgagcatgcctgccagctccttgtcctgacaccagtaacattaacctgtggaatatcttgaggaacaacattggtaaagacctgtctaaagtctctatgcctgtggagctaaacgagccgctcaacaccctgcagcacctctgtgaggaaatggaatacagcgagctcctggacaaggcttcggaaactgatgatccatatgagcgcatggttctcgttgccgcatttgcagtttcaggatactgctccacctatttcagagcaggaagtaagccattcaacccagtccttggggagacttatgaatgcattagagaagacaagggattccgctttttctcagaacaggttagccatcatccacccatttctgcctgtcactgtgaatcaaagaattttgtgttttggcaagatatcagatggaaaaacaagttctgggggaagtcgatggaaatcctgcctgttggaacactgaatgtcatgcttccaaagtatggagattactatgtgtggaataaagtcaccacttgcatacacaacatcctcagtgggagaagatggatagaacattatggagaagtaaccatcagaaataccaaaagcagtgtttgcatttgcaaactcacatttgtcaaggtgaattattggaattctaacatgaatgaagtccagggggtggtgatagatcaggaggggaaggcggtgtaccggctgtttggaaagtggcatgaaggactctactgtggtgtggccccctctgcaaagtgcatttggagaccaggttccatgccaacaaactatgagctgtactatggcttcacaaggtttgctattgagctcaatgagttagatccagtactaaaagatctccttccaccaacagacgcccggttccggccagatcaaagatttttggaagaaggaaatttagaagctgcagcatcagagaagcaaagagtagaggaactccagagatctcggagacgatatatggaagaaaacaatcttgaacatataccaaaattttttaaaaaagttattgatgccaatcaaagagaagcctgggtttctaacgacacctactgggagcttcgaaaggaccctgggtttagcaaagtagacagccctgttctttggtag

SEQ ID NO: 16 - DNA, 840 bp, Homo Sapiens
atgcctgcttttattcaaatgggcagagataaaaacttctcgagtctccacactgtcttttgtgccactggaggtggagcgtacaaatttgagcaggattttctcacaataggtgatcttcagctttgcaaactggatgaactagattgcttgatcaaaggaattttatacattgactcagtcggattcaatggacggtcacagtgctattactttgaaaaccctgctgattctgaaaagtgtcagaagttaccatttgatttgaaaaatccgtatcctctgcttctggtgaacattggctcaggggttagcatcttagcagtatattccaaagataattacaaacgggtcacaggtactagtcttggaggaggaactttttttggtctctgctgtcttcttactggctgtaccacttttgaagaagctcttgaaatggcatctcgtggagatagcaccaaagtggataaactagtacgagatatttatggaggggactatgagaggtttggactgccaggctgggctgtggcttcaagctttggaaacatgatgagcaaggagaagcgagaggctgtcagtaaagaggacctggccagagcgactttgatcaccatcaccaacaacattggctcaatagcaagaatgtgtgcccttaatgaaaacattaaccaggtggtatttgttggaaatttcttgagaattaatacgatcgccatgcggcttttggcatatgctttggattattggtccaaggggcagttgaaagcacttttttcggaacacgagggttattttggagctgttggagcactccttgagctgttgaagatcccgtga

SEQ ID NO: 17 - DNA, 1011 bp, Homo Sapiens
atgctgcagtccctggccggcagctcgtgcgtgcgcctggtggagcggcaccgctcggcctggtgcttcggcttcctggtgctgggctacttgctctacctggtcttcggcgcagtggtcttctcctcggtggagctgccctatgaggacctgctgcgccaggagctgcgcaagctgaagcgacgcttcttggaggagcacgagtgcctgtctgagcagcagctggagcagttcctgggccgggtgctggaggccagcaactacggcgtgtcggtgctcagcaacgcctcgggcaactggaactgggacttcacctccgcgctcttcttcgccagcaccgtgctctccaccacaggttatggccacaccgtgcccttgtcagatggaggtaaggccttctgcatcatctactccgtcattggcattcccttcaccctcctgttcctgacggctgtggtccagcgcatcaccgtgcacgtcacccgcaggccggtcctctacttccacatccgctggggcttctccaagcaggtggtggccatcgtccatgccgtgctccttgggtttgtcactgtgtcctgcttcttcttcatcccggccgctgtcttctcagtcctggaggatgactggaacttcctggaatccttttatttttgttttatttccctgagcaccattggcctgggggattatgtgcctggggaaggctacaatcaaaaattcagagagctctataagattgggatcacgtgttacctgctacttggccttattgccatgttggtagttctggaaaccttctgtgaactccatgagctgaaaaaattcagaaaaatgttctatgtgaagaaggacaaggacgaggatcaggtgcacatcatagagcatgaccaactgtccttctcctcgatcacagaccaggcagctggcatgaaagaggaccagaagcaaaatgagccttttgtggccacccagtcatctgcctgcgtggatggccctgcaaaccattga

SEQ ID NO: 18 - DNA, 1719 bp, Homo Sapiens
atggacgccctgtgcggttccggggagctcggctccaagttctgggactccaacctgtctgtgcacacagaaaacccggacctcactccctgcttccagaactccctgctggcctgggtgccctgcatctacctgtgggtcgccctgccctgctacttgctctacctgcggcaccattgtcgtggctacatcatcctctcccacctgtccaagctcaagatggtcctgggtgtcctgctgtggtgcgtctcctgggcggaccttttttactccttccatggcctggtccatggccgggcccctgcccctgttttctttgtcacccccttggtggtgggggtcaccatgctgctggccaccctgctgatacagtatgagcggctgcagggcgtacagtcttcgggggtcctcattatcttctggttcctgtgtgtggtctgcgccatcgtcccattccgctccaagatccttttagccaaggcagagggtgagatctcagaccccttccgcttcaccaccttctacatccactttgccctggtactctctgccctcatcttggcctgcttcagggagaaacctccatttttctccgcaaagaatgtcgaccctaacccctaccctgagaccagcgctggctttctctcccgcctgtttttctggtggttcacaaagatggccatctatggctaccggcatcccctggaggagaaggacctctggtccctaaaggaagaggacagatcccagatggtggtgcagcagctgctggaggcatggaggaagcaggaaaagcagacggcacgacacaaggcttcagcagcacctgggaaaaatgcctccggcgaggacgaggtgctgctgggtgcccggcccaggccccggaagccctccttcctgaaggccctgctggccaccttcggctccagcttcctcatcagtgcctgcttcaagcttatccaggacctgctctccttcatcaatccacagctgctcagcatcctgatcaggtttatctccaaccccatggccccctcctggtggggcttcctggtggctgggctgatgttcctgtgctccatgatgcagtcgctgatcttacaacactattaccactacatctttgtgactggggtgaagtttcgtactgggatcatgggtgtcatctacaggaaggctctggttatcaccaactcagtcaaacgtgcgtccactgtgggggaaattgtcaacctcatgtcagtggatgcccagcgcttcatggaccttgcccccttcctcaatctgctgtggtcagcacccctgcagatcatcctggcgatctacttcctctggcagaacctaggtccctctgtcctggctggagtcgctttcatggtcttgctgattccactcaacggagctgtggccgtgaagatgcgcgccttccaggtaaagcaaatgaaattgaaggactcgcgcatcaagctgatgagtgagatcctgaacggcatcaaggtgctgaagctgtacgcctgggagcccagcttcctgaagcaggtggagggcatcaggcagggtgagctccagctgctgcgcacggcggcctacctccacaccacaaccaccttcacctggatgtgcagccccttcctggtgaggcttggcacagggctgggtccctgcctccagggctctgggtgcccaggcatggccagggctcattggactctaccctga

SEQ ID NO: 19 - DNA, 502 bp, Homo Sapiens
agcccatgatgccgtggcacaggaaggccaatgtcgggtagatgacaaggtgaatttccattttattctgtttaacaacgtggatggccacctctatgaacttgatggacgaatgccttttccggtgaaccatggcgccagttcagaggacaccctgctgaaggacgctgccaaggtgtgcagagaattcaccgagcgtgagcaaggagaagtccgcttctctgccgtggctctctgcaaggcagcctaatgctctgtgggagggactttgctgatttcccctcttcccttcaacatgaaaatatataccccccatgcagtctaaaatgcttcagtacttgtgaaacacagctgttcttctgttctgcagacacgccttcccctcagccacacccaggcacttaagcacaagcagagtgcacagctgtccactgggccattgtggtgtgagcttcagatggtgaagcattctccccagtgtatgtcttgtatccgatatcta

SEQ ID NO: 20 - DNA, 80 bp, Homo Sapiens
atcttcgaaatgcttgaaggagtgcaaggacctactgcagtcaggaagcgattttttgaatccatcatcaaggaagcagc

SEQ ID NO: 21 - DNA, 550 bp, Homo Sapiens
tcctatgtcttctttcttaaatccagttgctgattttgtaaaatacagttgtgataaagcagcattacgggggggaaaaagctatattccaactggtgttaaatgtattcaacaaaatcttacatcatacagtatttatttcttaattaatagaacttcagtgatatacttggtagatatctcaagccttttgtcttttacacaatggtgctctatcctattgttttcttttcaaagaagcatctgaacacttgcatttctattttcctatccaaaggcatccacatctaagtgtgtttttaaagttgattaaaattatttttctgttaaagcattctgaaagtgtttgtctttacctagaatgatttgtacacactcgtggtcaactgaacatgaatgtcagtagtagtctaattatgggaagggtaaacgtgttagattaaggctcttaaagctctaaaccatataaactatggacttgtatcatgatttaactgttcttagatctttcttacacagtgattcattcctctatttgtacagtggcttt

SEQ ID NO: 22 - DNA, 404 bp, Homo Sapiens
ggaccctgaactcagactctacagattgccctccaagtgaggacttggctcccccactccttcgacgcccccacccccgccccccgtgcagagagccggctcctgggcctgctggggcctctgctccagggcctcagggccggcctggcagccggggagggccggagcggagggcgcgccttggccccacaccaacccccagggcctccccgcagtccctgcctagcccctctgccccagcaaatgcccagcccaggcaaattgtatttaaagaatcctgggggtcattatggcattttacaaactgtgaccgtttctgtgtgaagatttttagctgtatttgtggtctctgtatttatatttatgtttagcaccgtcagtgttcctatccaatttcaaaaaag

SEQ ID NO: 23 - DNA, 318 bp, Homo Sapiens
tgttcactggtacgttggggaggggatggaggaaggtgagttttcagaggcccgtgaggacatggctgcccttgagaaggattatgaggaggttggtgtggattctgttgaaggagagggtgaggaagaaggagaggaatactaaagttaaaacgtcacaaaggtgctgcttttacagggaagcttattctgttttaaacattgaaaagttgtggtctgatcagttaatttgtatgtagcagtgtatgctctcatatacaattactgacctatgctctaaaacatgaatgctttgttacagacccaagctgtccattt

SEQ ID NO: 24 - DNA, 545 bp, Homo Sapiens
  misc_feature (133)..(133): n is a, c, g, or t
tattcatggcttttgcatcctatgcaacaattattctttcaaaaatgatgcttatgagtactgcaactgcattctatagattgacaagaaaggtttttgccaatccagaagactgtgtagcatttggcaaagnagaaaatgccaagaagtatcttcgaacagatgacagagtagaacgtgtacgcagagcccacctgaatgaccttgaaaatattattccatttcttggaattggcctcctgtattccttgagtggtcccgacccctctacagccatcctgcacttcagactatttgtcggagcacggatctaccacaccattgcatatttgacaccccttccccagccaaatagagctttgagtttttttgttggatatggagttactctttccatggcttacaggttgctgaaaagtaaattgtncctgtaaagttataatgaatactttcttagattttaggtaggnngggngnagaggaatntanganacnttnnggntannnnnaacccacttttgatattagcatttgccatattcctg

SEQ ID NO: 25 - DNA, 228 bp, Homo Sapiens
  misc_feature (55)..(55): n is a, c, g, or t
gaaaaaatggttgacctcacccaggtaatggatgatgaagtattcatggcttttncatcctatgcaacaattatnctttcaaaaatnatgcttatgagtactgcaactgcattctatagattgacaagaaaggtttttgccaatccagaagactgtgtagcatttggcaaaggagaaaatgccaagaagtatcttcgaacagatgacagagtagaacgtgtacgcaga

SEQ ID NO: 26 - DNA, 65 bp, Homo Sapiens
ggaaagcatgtctgctgggtgtgaccatgtttcctctcaataaagttcccctgtgacactcaaaa

SEQ ID NO: 27 - DNA, 481 bp, Homo Sapiens
ggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaattttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtat

SEQ ID NO: 28 - DNA, 302 bp, Homo Sapiens
gactttatacttaacatcagatcttttctataatatcctactactttggttttcctagctccataccacacacctaaacctgtattatgaattacatattacaaagtcataaatgtgccatatggatatacagtacattctagttggaatcgtttactctgctagaatttaggtgtgagattttttgtttcccaggtatagcaggcttatgtttggtggcattaaattggtttctttaaaatgctttggtggcacttttgtaaacagattgcttctagattgttacaaaccaagcctaagac

SEQ ID NO: 29 - DNA, 672 bp, Homo Sapiens
atgcagctcaagccgatggagatcaaccccgagatgctgaacaaagtgctgtcccggctgggggtcgccggccagtggcgcttcgtggacgtgctggggctggaagaggagtctctgggctcggtgccagcgcctgcctgcgcgctgctgctgctgtttcccctcacggcccagcatgagaacttcaggaaaaagcagattgaagagctgaagggacaagaagttagtcctaaagtgtacttcatgaagcagaccattgggaattcctgtggcacaatcggacttattcacgcagtggccaataatcaagacaaactgggatttgaggatggatcagttctgaaacagtttctttctgaaacagagaaaatgtcccctgaagacagagcaaaatgctttgaaaagaatgaggccatacaggcagcccatgatgccgtggcacaggaaggccaatgtcgggtagatgacaaggtgaatttccattttattctgtttaacaacgtggatggccacctctatgaacttgatggacgaatgccttttccggtgaaccatggcgccagttcagaggacaccctgctgaaggacgctgccaaggtctgcagagaattcaccgagcgtgagcaaggagaagtccgcttctctgccgtggctctctgcaaggcagcctaa

SEQ ID NO: 30 - DNA, 570 bp, Homo Sapiens
atgaccgataaaacagagaaggtggctgtagatcctgaaactgtgtttaaacgtcccagggaatgtgacagtccttcgtatcagaaaaggcagaggatggccctgttggcaaggaaacaaggagcaggagacagccttattgcaggctctgccatgtccaaagcaaagaagcttatgacaggacatgctattccacccagccaattggattctcagattgatgacttcactggtttcagcaaagataggatgatgcagaaacctggtagcaatgcacctgtgggaggaaacgttaccagcagtttctctggagatgacctagaatgcagagaaacagcctcctctcccaaaagccaacaagaaattaatgctgatataaaacgtaaattagtgaaggaactccgatgcgttggacaaaaatatgaaaaaatcttcgaaatgcttgaaggagtgcaaggacctactgcagtcaggaaacgattttttgaatccatcatcaaggaagcagcaagatgtatgagacgagactttgttaagcaccttaagaagaaactgaaacgtatgatttga

SEQ ID NO: 31 - DNA, 1023 bp, Homo Sapiens
atgagcgaactggaacagttgaggcaagaagcagaacaactgcggaatcagattcaggatgctcggaaagcatgtaatgatgcaacgcttgttcagattacatcaaatatggactctgtgggtcgaatacaaatgcgaacaagacgtacactgaggggccacctagctaaaatctatgctatgcattggggatacgattccaggctgctagtcagtgcttctcaagatggaaaattaattatttgggatagctatacaacaaataagatgcatgctattcctttgaggtcctcctgggtgatgacctgtgcttatgctccctctggtaattatgttgcctgtggaggcttggacaacatctgctctatatataacttaaagaccagagagggaaatgtgagagtaagccgagagttgccaggtcacacagggtacttgtcctgctgtcgttttttagatgacagccaaattgttacaagttcaggagatacaacttgtgctttatgggacatcgaaactgcccagcagaccaccacattcactgggcattctggagatgtgatgagtctttctttgagtcctgacatgaggacttttgtttctggtgcttgtgatgcctcttccaaattatgggatattcgagatggaatgtgtagacagtctttcacgggacatgtctcagatatcaatgctgtcagttttttcccaaatggatatgccttcgccactggctctgatgatgccacttgccggctctttgaccttcgtgcagatcaagagttattattgtattctcatgacaatatcatctgtggaatcacttctgtagccttctcaaaaagtgggcgtctcttgttggctggttacgatgactttaattgtaatgtatgggacacgctaaaaggagatcgtgcaggtgtccttgctggtcatgacaaccgtgtgagctgcttaggtgtaactgatgatggcatggctgtggcaacaggctcttgggacagttttcttagaatctggaattaa

SEQ ID NO: 32 - DNA, 795 bp, Homo Sapiens
atgatcatgagctcgtatttgatggactctaactacatcgatccgaaatttcctccatgcgaagaatattcgcaaaatagctacatccctgaacacagtccggaatattacggccggaccagggaatcgggattccagcatcaccaccaggagctgtacccaccaccgcctccgcgccctagctaccctgagcgccagtatagctgcaccagtctccaggggcccggcaattcgcgaggccacgggccggcccaggcgggccaccaccaccccgagaaatcacagtcgctctgcgagccggcgcctctctcaggcgcctccgcctccccgtccccagccccgccagcctgcagccagccagcccccgaccatccctccagcgccgccagcaagcaacccatagtctacccatggatgaaaaaaattcacgttagcacggtgaaccccaattataacggaggggaacccaagcgctcgaggacagcctatacccggcagcaagtcctggaattagagaaagagtttcattacaaccgctacctgacccgaaggagaaggatcgagatcgcccactcgctgtgcctctctgagaggcagatcaaaatctggttccaaaaccgtcgcatgaaatggaagaaggaccaccgactccccaacaccaaagtcaggtcagcacccccggccggcgctgcgcccagcaccctttcggcagctaccccgggtacttctgaagaccactcccagagcgccacgccgccggagcagcaacgggcagaggacattaccaggttataa

SEQ ID NO: 33 - DNA, 1356 bp, Homo Sapiens
atgcgtgagtgcatctccatccacgttggccaggctggtgtccagattggcaatgcctgctgggagctctactgcctggaacacggcatccagcccgatggccagatgccaagtgacaagaccattgggggaggagatgattccttcaacaccttcttcagtgagacgggggctggcaagcatgtgccccgggcagtgtttgtagacttggaacccacagtcattgatgaagttcgcactggcacctaccgccagctcttccaccctgagcaacttatcacaggcaaagaagatgctgccaataactatgcccgagggcactacaccattggcaaggagatcattgacctcgtgttggaccgaattcgcaagctggccgaccagtgcacgggtctccagggcttcttggttttccacagctttggtgggggaactggttctgggttcacctcgctgctcatggaacgtctctcagttgattatggcaagaagtccaagctggagttctctatttacccggcgccccaggtttccacagctgtagttgagccctacaactccatcctcaccacccacaccaccctggagcactctgattgtgccttcatggtagacaatgaggccatctatgacatctgtcgtagaaacctcgatattgagcgtccaacctatactaacctgaataggttaataggtcaaattgtgtcctccatcactgcttccctgagatttgatggagccctgaatgttgacctgacagaattccagaccaacctggtgccctatccccgcatccacttccctctggccacatatgcccctgtcatctctgctgagaaagcctaccatgaacagctttctgtagcagagatcaccaatgcttgctttgagccagccaaccagatggtgaaatgtgaccctcgccatggtaaatacatggcttgctgcctgttgtaccgtggtgacgtggttcccaaagatgtcaatgctgccattgccaccatcaagaccaagcgtaccatccagtttgtggattggtgccccactggcttcaaggttggcatcaactaccagcctcccactgtggtgcctggtggagacctggccaaggtacagagagctgtgtgcatgctgagcaacaccacagccattgctgaggcctgggctcgcctggaccacaagtttgacctgatgtatgccaaacgtgcctttgttcactggtacgttggggaggggatggaggaaggtgagttttcagaggcccgtgaggacatggctgcccttgagaaggattatgaggaggttggtgtggattctgttgaaggagagggtgaggaagaaggagaggaatactaa

SEQ ID NO: 34 - DNA, 468 bp, Homo Sapiens
atggttgacctcacccaggtaatggatgatgaagtattcatggcttttgcatcctatgcaacaattattctttcaaaaatgatgcttatgagtactgcaactgcattctatagattgacaagaaaggtttttgccaatccagaagactgtgtagcatttggcaaaggagaaaatgccaagaagtatcttcgaacagatgacagagtagaacgtgtacgcagagcccacctgaatgaccttgaaaatattattccatttcttggaattggcctcctgtattccttgagtggtcccgacccctctacagccatcctgcacttcagactatttgtcggagcacggatctaccacaccattgcatatttgacaccccttccccagccaaatagagctttgagtttttttgttggatatggagttactctttccatggcttacaggttgctgaaaagtaaattgtacctgtaa

SEQ ID NO: 35 - DNA, 468 bp, Homo Sapiens; identical to SEQ ID NO: 34

SEQ ID NO: 36 - DNA, 747 bp, Homo Sapiens
atggagagagccagtctgatccagaaggccaagctggcagagcaggccgaacgctatgaggacatggcagccttcatgaaaggcgccgtggagaagggcgaggagctctcctgcgaagagcgaaacctgctctcagtagcctataagaacgtggtgggcggccagagggctgcctggagggtgctgtccagtattgagcagaaaagcaacgaggagggctcggaggagaaggggcccgaggtgcgtgagtaccgggagaaggtggagactgagctccagggcgtgtgcgacaccgtgctgggcctgctggacagccacctcatcaaggaggccggggacgccgagagccgggtcttctacctgaagatgaagggtgactactaccgctacctggccgaggtggccaccggtgacgacaagaagcgcatcattgactcagcccggtcagcctaccaggaggccatggacatcagcaagaaggagatgccgcccaccaaccccatccgcctgggcctggccctgaacttttccgtcttccactacgagatcgccaacagccccgaggaggccatctctctggccaagaccactttcgacgaggccatggctgatctgcacaccctcagcgaggactcctacaaagacagcaccctcatcatgcagctgctgcgagacaacctgacactgtggacggccgacaacgccggggaagaggggggcgaggctccccaggagccccagagctga

SEQ ID NO: 37 - DNA, 2649 bp, Homo Sapiens
atgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgctggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactag

SEQ ID NO: 38 - DNA, 531 bp, Homo Sapiens
atgtcggccggcggagcgtcagtcccgccgcccccgaaccccgccgtgtccttcccgccgccccgggtcaccctgcccgccggccccgacatcctgcggacctactcgggcgccttcgtctgcctggagattctgttcgggggtcttgtctggattttggttgcctcctccaatgttcctctacctctactacaaggatgggtcatgtttgtgtccgtgacagcgtttttcttttcgctcctctttctgggcatgttcctctctggcatggtggctcaaattgatgctaactggaacttcctggattttgcctaccattttacagtatttgtcttctattttggagcctttttattggaagcagcagccacatccctgcatgatttgcattgcaatacaaccataaccgggcagccactcctgagtgataaccagtataacataaacgtagcagcctcaatttttgcctttatgacgacagcttgttatggttgcagtttgggtctggctttacgaagatggcgaccgtaa

SEQ ID NO: 39 - DNA, 507 bp, Homo Sapiens
aaatgctcacagaccggcggtctacagaccttaatgagagccgccgtgcagacgtgcttgccttcccaagctctggcttcactgacttggcagagattgtgtcccggattgagccccccacgagctatgtctctgatggctgtgctgacggagaggagtcagattgtctgacagagtatgaggaggacgccggacccgactgctcgagggatgaaggggggtcccccgagggcgcaagtcccagcactgcctccgagatggaggaggagaagtcgattctccggcaacgacgctgtctgccccaggagccgcccggctcagccacagatgcctgaggacctcgacaggggtcaccccctccctcccacccctggactgggctgggggtggccccgtgggggtagctcactccccctcctgctgctatgcctgtgacccccgcggcccacacactggactgacctgccctgagcggggatgcagtgttgcactgatgacttgaccagc

SEQ ID NO: 40 - DNA, 481 bp, Homo Sapiens
  misc_feature (174)..(176): n is a, c, g, or t
tttaagtcccatcttcccctgggcataggccatagggatagaagttaaagttcttgagcttattcagaagctggatctgcaatctgaatgctactcataacataacaaaatagtatgttaaacagctcttaaatcttactggcttaccacattaaatgatttctctctcctaannnannnnaaannggnagccatccatgggatgagtcagaggttcagactcttccagtctgtagctctaccttctcttagggtacttagatggatcccctgttctacaaactgccagtcagcaagggaagaaaaagggcagcaatgaccctcaatgggccatttgagggatctggcctggaaatgggcttcctctcttcttctcacacctcactggctggaaacagtcacatgaccccagtcacnnnnnnnnnnnnnnnnnnnnnnnnnnnnnnnnnnnnnnnaagggcaaagctgtttaagggccact

SEQ ID NO: 41 - DNA, 532 bp, Homo Sapiens
ggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatggacactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttaggacaccaggctgcagatgatcagggatccaata

SEQ ID NO: 42 - DNA, 123 bp, Homo Sapiens
gtaccttgtggtatctgactctggcatctcaactgactacagctcaggggactcccagggagcccaagggggcttatccgatggcccctactccaacccttatgagaacagccttatcccagc

SEQ ID NO: 43 - DNA, 288 bp, Homo Sapiens
  misc_feature (175)..(175): n is a, c, g, or t
gaaagcatgtggtcaatttctgctgctcggggtttgagaagttctatattaggatgtcatttacatacttttcaacagaagattgaggacactgaaatatgaggtgtgtgaagaaagtacacagagaaacttttgtggttgacttataaaacttgtaagtgctgcatgagatagntgttcagagtaatagtcttgtgcattattcaaattttagtaaattgttttaccaattgcacattagatagtatagtggattttgtaatgcttttcttagactgtgtgaactta

SEQ ID NO: 44 - DNA, 495 bp, Homo Sapiens
  misc_feature (379)..(383): n is a, c, g, or t
gagtattctccaaatcacttgactttccttttctcagagttatagttctccagagatgtcaccagatgattcacattaaaggtacatttccagttgacttttttatttcagatattagtaaatgattaccagagaactaattcaaactgtcatggttggaaatactctgatgctcaactccctaatctatagagtgagttaaggtctatactcttttgcaagcattgttctagtggcagtgaccctgttcatgattcagcctgacatttactatgctgccctgaaaaccaactagatgggagcttgtttctctctgcattgtgtagtgagtggtgtcacccaggactttcatgtgagaaaaagcaccttgcttttaaannnnnaannnnnagtgttttttgtaatgcctcagtttattcttatttatggttgattatatgtgatttagaacacttgctatttatgtctgatcccacaaatcagcactcactttat

SEQ ID NO: 45 - DNA, 520 bp, Homo Sapiens
agccatttgaagacgcctcgtttgcgctgcggacgggggagatgagcgggcccgtgttcacggattccggcatccacatcatcctccgcactgagtgagggtggggagcccaggcctggcctcggggcagggcagggcggctaggccggccagctcccccttgcccgccagccagtggccgaaccccccactccctgccaccgtcacacagtatttattgttcccacaatggctgggagggggcccttccagattgggggccctggggtccccactccctgtccatccccagttggggctgcgaccgccagattctcccttaaggaattgacttcagcaggggtgggaggctcccagacccagggcagtgtggtgggaggggtgttccaaagagaaggcctggtcagcagagccgccccgtgtccccccaggtgctggaggcagactcgagggccgaattgtttctagttaggccacgctcctctgttcagtcgcaaaggtgaacactcatgcggcagcc

SEQ ID NO: 46 - DNA, 420 bp, Homo Sapiens
  misc_feature (188)..(188): n is a, c, g, or t
ccagcaggggcgtcgttttgttgccattttgttgaacgttatgggtttatgggtgttcctggaacttgtctttgtgcattcgttgctgtttgtgttaccctcactgtccccatgtcccacccacgtcctacggcactcaggaagcacttggtgaggacgagccctcacccttcttgtcttccttcccngcangcgcccgcagcgggccatttacacgtcgaggctggcacctggcgcgctcgggggccactgtagcgtctgcctgctccctggactcgcaggcctcncctgtggcgccttcccagggccagcctgggtcacgagatgctgtcactcagccagatcagtattgacccaccaggggaggtggggtttggtgagagacgccagcctcagactttttcccactgagggtccaga

SEQ ID NO: 47 - DNA, 487 bp, Homo Sapiens
aagaaccagtgtcaatccgcagaccctctgtgaagccaggccggccgggccgagccagcagcccctctccctagactcagaggcgccgcggggaggggtggccccgccgaggcttcaggggccccctccccaccaaagggttcacctcacacttgaatgtacaacccaccccactgtcgggaaggcctccgtcctcggcccctgcctcttgctgctgtcctgtccccgagcccctgcaggtccccccccgcccccccactcaagagttagagcaggtggctgcaggccttgggcccggagggaaggccactgccggccacttggggcagacacagacacctcaaggatctgtcacggaaggcgtcctttttccttgtagctaacgttaggcctgagtagctcccctccatccttgtagacgctccagtccctactactgtgacggcatttccatccctcccctgcccgggaagggaccttgcaggga

SEQ ID NO: 48 - DNA, 86 bp, Homo Sapiens
ccgccgctacgaatacgatcactgggacgcggccatccacggcttccgagagacagagaagtcgcgctggtcagaagccagccggg

SEQ ID NO: 49 - DNA, 4128 bp, Homo Sapiens
atggaggctccgctgcaaactggaatgatggggacatcgagtcacgggctggctacgaactcctcgggggcgaaggtggcggagagggatgggttccaggacgtcctggcgcccggggaaggctcggcgggacggatttgcggtgcgcagccagtgccgttcgtccctcaggtgcttggcgtgatgatcggggccggagtggcggtggtggtcacggccgtgctcatcctcctggtggtgcggaggctgcgagtgccaaaaaccccagccccggatggcccccggtatcggttccggaagagggacaaagtgctcttctatggccggaagattatgcggaaggtgtcacaatccacctcctccctcgtggatacctctgtctccgccacctcccggccacgcatgaggaagaaactgaagatgctcaacattgccaagaagatcctgcgcatccagaaagagacgcccacgctgcagcggaaggagcccccgcccgcagtgctagaagctgacctgaccgagggcgacctggctaactcccatctgccctctgaagtgctttatatgctcaagaacgtccgggtgctgggccacttcgagaagccactcttcctggagctctgccgccacatggtcttccagcggctgggccagggtgactacgtcttccggccgggccagccagatgccagcatctacgtggtgcaggacgggctgctggagctctgtctgccagggcctgacgggaaggagtgtgtggtgaaggaagtggttcctggggacagcgtcaacagccttctcagcatcctggatgtcatcaccggtcaccagcatccccagcggaccgtgtctgcccgggcggcccgggactccacggtgctgcgcctgccggtggaagcattctccgcggtcttcaccaagtacccggagagcttggtgcgggtcgtgcagatcatcatggtgcggctgcagcgagtcaccttcctggcactgcacaactacctgggtctgaccaatgagctcttcagccacgagatccagcccctgcgtctgttccccagccccggcctcccaactcgcaccagccctgtgcggggctccaagagaatggtcagcacctcagctacagacgagcccagggagaccccagggcggccacccgatcccaccggggccccgctgcctggacctacaggggaccctgtgaagcccacatccctggaaaccccctcggcccctctgctgagccgctgcgtctccatgccaggggacatctcaggcttgcagggtggcccccgctccgacttcgacatggcctatgagcgtggccggatctccgtgtccctgcaggaagaggcctccggggggtccctggcagcccccgctcggacccccactcaggagcctcgtgagcagccggcaggcgcctgtgaatacagctactgtgaggatgagtcggccactggtggctgccctttcgggccctaccagggccgccagaccagcagcatcttcgaggcagcaaagcaggagctggccaagctgatgcggattgaggacccctccctcctgaacagcagagtcttgctgcaccacgccaaagctggcaccatcattgcccgccagggagaccaggacgtgagcctgcacttcgtgctctggggctgcctgcacgtgtaccagcgcatgatcgacaaggcggaggacgtgtgcctgttcgtagcgcagcccggggaactggtggggcagctggcggtgctcactggcgaacctctcatcttcacactgcgagcccaacgcgactgcaccttcctgcggatctccaagtccgacttctatgagatcatgcgcgcacagcccagtgtggtgctgagtgcggcgcacacggtggcagccaggatgtcgcccttcgtgcgccagatggacttcgccatcgactggactgcagtggaggcgggacgcgcgctgtacaggcagggcgaccgctccgactgcacttacatcgtgctcaatgggcggctgcgtagcgtgatccagcgaggcagtggcaagaaggagctggtgggcgagtacggccgcggcgacctcatcggcgtggtggaggcactgacccggcagccgcgagccacgacggtgcacgcggtgcgcgacacggagctggccaagcttcccgagggcaccttgggtcacatcaaacgccggtacccgcaggtcgtgacccgccttatccacctactgagccagaaaattctagggaatttgcagcagctgcaaggacccttcccagcaggctctgggttgggtgtgcccccacactcggaactcaccaacccagccagcaacctggcaactgtggcaatcctgcctgtgtgtgctgaggtccccatggtggccttcacgctggagctgcagcacgccctgcaggccatcggtccgacgctactccttaacagtgacatcatccgggcacgcctgggggcctccgcactggatagcatccaagagttccggctgtcagggtggctggcccagcaggaggatgcacaccgtatcgtactctaccagacggacgcctcgctgacgccctggaccgtgcgctgcctgcgacaggccgactgcatcctcattgtgggcctgggggaccaggagcctaccctcggccagctggagcagatgctggagaacacggctgtgcgcgcccttaagcagctagtcctgctccaccgagaggagggcgcgggccccacgcgcaccgtggagtggctaaatatgcgcagctggtgctcggggcacctgcacctgcgctgtccgcgccgcctcttttcgcgccgcagccctgccaagctgcatgagctctacgagaaggttttctccaggcgcgcggaccggcacagcgacttctcccgcttggcgagggtgctcacggggaacaccattgcccttgtgctaggcgggggcggggccaggggctgctcgcacatcggagtactaaaggcattagaggaggcgggggtccccgtggacctggtgggcggcacgtccattggctctttcatcggagcgttgtacgcggaggagcgcagcgccagccgcacgaagcagcgggcccgggagtgggccaagagcatgacttcggtgctggaacctgtgttggacctcacgtacccagtcacctccatgttcactgggtctgcctttaaccgcagcatccatcgggtcttccaggataagcagattgaggacctgtggctgccttacttcaacgtgaccacagatatcaccgcctcagccatgcgagtccacaaagatggctccctgtggcggtacgtgcgcgccagcatgacgctgtcgggctacctgcccccgctgtgcgaccccaaggacgggcacctactcatggatggcggctacatcaacaatctgccagcggacatcgcccgcagcatgggtgccaaaacggtcatcgccattgacgtggggagccaggatgagacggacctcagcacctacggggacagcctgtccggctggtggctgctgtggaagcggctgaatccctgggctgacaaggtaaaggttccagacatggctgaaatccagtcccgcctggcctacgtgtcctgtgtgcggcagctagaggttgtcaagtccagctcctactgcgagtacctgcgcccgcccatcgactgcttcaagaccatggactttgggaagttcgaccagatctatgatgtgggctaccagtacgggaaggcggtgtttggaggctggagccgtggcaacgtcattgagaaaatgctcacagaccggcggtctacagaccttaatgagagccgccgtgcagacgtgcttgccttcccaagctctggcttcactgacttggcagagattgtgtcccggattgagccccccacgagctatgtctctgatggctgtgctgacggagaggagtcagattgtctgacagagtatgaggaggacgccggacccgactgctcgagggatgaaggggggtcccccgagggcgcaagccccagcactgcctccgagatggaggaggagaagtcgattctccggcaacgacgctgtctgccccaggagccgcccggctcagccacagatgcctga

SEQ ID NO: 50 - DNA, 1527 bp, Homo Sapiens
atggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatggacactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttag

SEQ ID NO: 51 - DNA, 1401 bp, Homo Sapiens
atgctagagaactacagaaacctggtctctctgggtcttactgtttctaagccagaactgattagccgtctggagcaaagacaggagccctggaatgtgaagagacatgagaccatagccaaacccccagctatgtcttctcattacactgaagaccttttgccagaacagtgcatgcaagattcattccaaaaagtgatactgagaagatatggaagctgtggacttgaggatttacacttaaggaaggatggggaaaatgtgggtgagtgtaaggatcaaaaagaaatttataatggacttaaccaatgtttgtcaactctacctagcaaaattttcccatataataaatgtgtgaaagtctttagtaaatcatcaaatctaaatagagaaaacataagacatactacagagaaacttttcaaatgtatgcaatgtggcaaagtctttaaatctcactcaggcctttcttatcataagataattcacactgaagagaaactctgcatatgtgaggaatgtggcaaaacctttaagtggttctcataccttactaaacataagagaattcacactggagagaaaccatacaaatgtgaagaatgtggcaaagcttttaactggtgctcgagtcttactaaacataagagaatccatactggtgagaaaccctacaaatgtgaagaatgtggaaaagcctttcactggtgttcaccctttgttagacataagaaaattcatacaggagaaaaaccctatacatgtgaagactgtggcagagcgtttaaccggcactcacatctcaccaaacataagacaattcacactggaaagaaaccctacaaatgtaaagaatgtgggaaagcctttaaccactgctcactacttactatacatgagagaacccatacgggagagaaaccctataaatgtgaagaatgtggcaaagcttttaactcatcatcaattcttactgaacataaggtaattcatagcggagagaaaccctacaaatgtgaaaaatgtgacaaagtctttaagaggttctcataccttactaaacacaagagaattcacactggagagaaaccctacaaatgtgaagaatgtggcaaagcttttaactggtcctcaatccttactgaacataagagaattcatactggagagaaaccctacaactgtgaagaatgtggaaaagcctttaatcggtgctcacaccttactagacataagaaaattcatactgccgtcaaacgctataaatgtgaagaatgtggcaaagcttttaaacggtgctcacatcttaatgaacataagagagttcaaagaggagagaaatcctgcaagtataaaaaatgtggggaagcttttaatcactgctcaaaccttactacgtaa

SEQ ID NO: 52 - DNA, 1539 bp, Homo Sapiens
atgctctttgagggcttggatctggtgtcggcgctggccaccctcgccgcgtgcctggtgtccgtgacgctgctgctggccgtgtcgcagcagctgtggcagctgcgctgggccgccactcgcgacaagagctgcaagctgcccatccccaagggatccatgggcttcccgctcatcggagagaccggccactggctgctgcagggttctggcttccagtcgtcgcggagggagaagtatggcaacgtgttcaagacgcatttgttggggcggccgctgatacgcgtgaccggcgcggagaacgtgcgcaagatcctcatgggcgagcaccacctcgtgagcaccgagtggcctcgcagcacccgcatgttgctgggccccaacacggtgtccaattccattggcgacatccaccgcaacaagcgcaaggtcttctccaagatcttcagccacgaggccctggagagttacctgcccaagatccagctggtgatccaggacacactgcgcgcctggagcagccaccccgaggccatcaacgtgtaccaggaggcgcagaagctgaccttccgcatggccatccgggtgctgctgggcttcagcatccctgaggaggaccttgggcacctctttgaggtctaccagcagtttgtggacaatgtcttctccctgcctgtcgacctgcccttcagtggctaccggcggggcattcaggctcggcagatcctgcagaaggggctggagaaggccatccgggagaagctgcagtgcacacagggcaaggactacttggacgccctggacctcctcattgagagcagcaaggagcacgggaaggagatgaccatgcaggagctgaaggacgggaccctggagctgatctttgcggcctatgccaccacggccagcgccagcacctcactcatcatgcagctgctgaagcaccccactgtgctggagaagctgcgggatgagctgcgggctcatggcatcctgcacagtggcggctgcccctgcgagggcacactgcgcctggacacgctcagtgggctgcgctacctggactgcgtcatcaaggaggtcatgcgcctgttcacgcccatttccggcggctaccgcactgtgctgcagaccttcgagcttgatggtttccagatccccaaaggctggagtgtcatgtatagcatccgggacacccatgacacagcgcccgtgttcaaagacgtgaacgtgttcgaccccgatcgcttcagccaggcgcggagcgaggacaaggatggccgcttccattacctcccgttcggtggcggtgtccggacctgcctgggcaagcacctggccaagctgttcctgaaggtgctggcggtggagctggctagcaccagccgctttgagctggccacacggaccttcccccgcatcaccttggtccccgtcctgcaccccgtggatggcctcagcgtcaagttctttggcctggactccaaccagaacgagatcctgccggagacggaggccatgctgagcgccacagtctaa

SEQ ID NO: 53 - DNA, 492 bp, Homo Sapiens
atggcggacgaggagaagctgccgcccggctgggagaagcgcatgagccgcagctcaggccgagtgtactacttcaaccacatcactaacgccagccagtgggagcggcccagcggcaacagcagcagtggtggcaaaaacgggcagggggagcctgccagggtccgctgctcgcacctgctggtgaagcacagccagtcacggcggccctcgtcctggcggcaggagaagatcacccggaccaaggaggaggccctggagctgatcaacggctacatccagaagatcaagtcgggagaggaggactttgagtctctggcctcacagttcagcgactgcagctcagccaaggccaggggagacctgggtgccttcagcagaggtcagatgcagaagccatttgaagacgcctcgtttgcgctgcggacgggggagatgagcgggcccgtgttcacggattccggcatccacatcatcctccgcactgagtga

SEQ ID NO: 54 - DNA, 1308 bp, Homo Sapiens
atgtccgccagcgccgtctacgtgctggacctgaagggcaaggtgctcatctgccggaactaccgtggcgacgtggacatgtcagaggtggagcacttcatgcccatcctgatggagaaggaggaggaggggatgctgtcgcccatcctggcccacgggggggtccgtttcatgtggatcaaacacaacaacctgtatctggttgccacatccaagaagaacgcgtgcgtgtcgctggtcttttctttcctctataaggtggtgcaggtgttttccgagtacttcaaggagctggaggaggagagcatccgggacaactttgttatcatctacgagctgctggacgagctcatggacttcggctacccccagaccaccgacagcaagatcctgcaggagtacatcactcaggaaggccacaagctggaaacaggggccccgcggccaccagccaccgtcaccaacgcggtgtcctggcggtccgaaggcatcaagtatcggaagaatgaggtgttcttggacgtcatcgagtctgtcaacctcttgggtaaatacccaggagtgggatggctgggtcacacggtcagcgccaacggcaatgtcctgcgcagcgagatcgtgggctccatcaagatgcgagtcttcctctcgggcatgcccgagctgcgcctgggcctcaacgacaaggtcctctttgacaacacgggccgcggcaaaagcaaatccgtggagctggaggatgtgaagttccaccagtgtgtgcggctatcacgcttcgagaatgaccgcaccatctccttcatcccacccgacggcgagttcgagctcatgtcctaccgtctcaacacccacgtcaagcctttgatatggatcgagtcggtgatcgagaagcactcccacagccgcatcgagtacatgatcaaggccaaaagccagttcaagcggcggtcaacagccaacaacgtggagatccacattcccgtgcccaatgatgccgactcacccaagttcaagacgacggtggggagcgttaagtgggtccccgagaacagcgagatcgtgtggtccatcaagtccttcccgggcggcaaggagtacctgatgcgggcccacttcggcctgcctagtgtggaggccgaagacaaggagggcaagcccccgatcagtgtcaagttcgagatcccttacttcactacctccggcatccaggtgcgctacctgaagatcattgagaagagtgggtaccaggccctgccctgggtgcgttatatcacgcagaatggagattaccagctccggacccagtga

SEQ ID NO: 55 - DNA, 1827 bp, Homo Sapiens
atggcagcggcggcggcggcggtggggccgggcgcgggcggcgcggggtcggcggtcccgggcggcgcggggccctgcgctaccgtgtcggtgttccccggcgcccgcctcctcaccatcggcgacgcgaacggcgagatccagcggcacgcggagcagcaggcgctgcgcctcgaggtgcgcgccggcccggactcggcgggcatcgccctctacagccatgaagatgtgtgtgtctttaagtgctcagtgtcccgagagacagagtgcagccgtgtgggcaagcagtccttcatcatcaccctgggctgcaacagcgtcctcatccagttcgccacacccaacgatttctgttccttctacaacatcctgaaaacctgccggggccacaccctggagcggtctgtgttcagcgagcggacggaggagtcttctgccgtgcagtacttccagttttatggctacctgtcccagcagcagaacatgatgcaggactacgtgcggacaggcacctaccagcgcgccatcctgcaaaaccacaccgacttcaaggacaagatcgttcttgatgttggctgtggctctgggatcctgtcgttttttgccgcccaagctggagcacggaaaatctacgcggtggaggccagcaccatggcccagcacgctgaggtcttggtgaagagtaacaacctgacggaccgcatcgtggtcatcccgggcaaggtggaggaggtgtcactccccgagcaggtggacatcatcatctcggagcccatgggctacatgctcttcaacgagcgcatgctggagagctacctccacgccaagaagtacctgaagcccagcggaaacatgtttcctaccattggtgacgtccaccttgcacccttcacggatgaacagctctacatggagcagttcaccaaggccaacttctggtaccagccatctttccatggagtggacctgtcggccctccgaggtgccgcggtggatgagtatttccggcagcctgtggtggacacatttgacatccggatcctgatggccaagtctgtcaagtacacggtgaacttcttagaagccaaagaaggagatttgcacaggatagaaatcccattcaaattccacatgctgcattcagggctggtccacggcctggctttctggtttgacgttgctttcatcggctccataatgaccgtgtggctgtccacagccccgacagagcccctgacccactggtaccaggtgcggtgcctgttccagtcaccactgttcgccaaggcaggggacacgctctcagggacatgtctgcttattgccaacaaaagacagagctacgacatcagtattgtggcccaggtggaccagaccggctccaagtccagtaacctcctggatctgaaaaaccccttctttagatacacgggcacaacgccctcacccccacccggctcccactacacatctccctcggaaaacatgtggaacacgggcagcacctacaacctcagcagcgggatggccgtggcagggatgccgaccgcctatgacttgagcagtgttattgccagtggctccagcgtgggccacaacaacctgattcctttagccaacacggggattgtcaatcacacccactcccggatgggctccataatgagcacggggattgtccaagggtcctccggcgcccagggcagtggtggtggcagcacgagtgcccactatgcagtcaacagccagttcaccatgggcggccccgccatctccatggcgtcgcccatgtccatcccgaccaacaccatgcactacgggagctag

SEQ ID NO: 56 - DNA, 666 bp, Homo Sapiens
atggccgggactgggctgctggcgctgcggacgctgccagggcccagctgggtgcgaggctcgggcccttccgtgctgagccgcctgcaggacgcggccgtggtgcggcctggcttcctgagcacggcagaggaggagacgctgagccgagaactggagcccgagctgcgccgccgccgctacgaatacgatcactgggacgcggccatccacggcttccgagagacagagaagtcgcgctggtcagaagccagccgggccatcctgcagcgcgtgcaggcggccgcctttggccccggccagaccctgctctcctccgtgcacgtgctggacctggaagcccgcggctacatcaagccccacgtggacagcatcaagttctgcggggccaccatcgccggcctgtctctcctgtctcccagcgttatgcggctggtgcacacccaggagccgggggagtggctggaactcttgctggagccgggctccctctacatccttaggggctcagcccgttatgacttctcccatgagatccttcgggatgaagagtccttctttggggaacgccggattccccggggccggcgcatctccgtgatctgccgctccctccctgagggcatggggccaggggagtctggacagccgcccccagcctgctga

SEQ ID NO: 57 - DNA, 741 bp, Homo Sapiens
atgacgacgggtgactgctgccacctccccggctccctgtgtgactgctccggcagccctgccttctccaaggtcgtggaggctacgggcctcggaccgccccagtatgtggcacaggtgacttcaagggatggccggctcctctccaccgtcatccgtgccttggacacaccgagtgatggtcctttctgccggatctgccatgagggagcgaacggggagtgcttgctgtccccgtgtggctgcaccggcacgctgggtgccgtgcataagagctgtctggagaagtggctttcctcatctaacaccagctactgcgagctgtgccacacggagtttgcagtggagaaacggcctcgacccctcacagagtggctgaaggacccggggccgcggacggagaagcggacactgtgctgcgacatggtgtgtttcctgttcatcacaccgctggccgccatctcaggctggttgtgcctgcgcggggcccaggaccacctccggctccacagccagctggaggccgtgggtctcattgccctcaccatcgccctcttcaccatctatgtcctctggacgctggtctccttccgctaccactgccagctgtactccgagtggagaaagaccaaccagaaagttcgcctgaagatccgggaggcggacagccccgagggcccccagcattctccactggcagctggactcctgaagaaggtggcagaggagacaccagtatga

SEQ ID NO: 58 - DNA, 6801 bp, Homo Sapiens
atggcccgcttcggagacgagatgccggcccgctacgggggaggaggctccggggcagccgccggggtggtcgtgggcagcggaggcgggcgaggagccgggggcagccggcagggcgggcagcccggggcgcaaaggatgtacaagcagtcaatggcgcagagagcgcggaccatggcactctacaaccccatccccgtccgacagaactgcctcacggttaaccggtctctcttcctcttcagcgaagacaacgtggtgagaaaatacgccaaaaagatcaccgaatggcctccctttgaatatatgattttagccaccatcatagcgaattgcatcgtcctcgcactggagcagcatctgcctgatgatgacaagaccccgatgtctgaacggctggatgacacagaaccatacttcattggaattttttgtttcgaggctggaattaaaatcattgcccttgggtttgccttccacaaaggctcctacttgaggaatggctggaatgtcatggactttgtggtggtgctaacgggcatcttggcgacagttgggacggagtttgacctacggacgctgagggcagttcgagtgctgcggccgctcaagctggtgtctggaatcccaagtttacaagtcgtcctgaagtcgatcatgaaggcgatgatccctttgctgcagatcggcctcctcctattttttgcaatccttatttttgcaatcatagggttagaattttatatgggaaaatttcataccacctgctttgaagaggggacagatgacattcagggtgagtctccggctccatgtgggacagaagagcccgcccgcacctgccccaatgggaccaaatgtcagccctactgggaagggcccaacaacgggatcactcagttcgacaacatcctgtttgcagtgctgactgttttccagtgcataaccatggaagggtggactgatctcctctacaatagcaacgatgcctcagggaacacttggaactggttgtacttcatccccctcatcatcatcggctccttttttatgctgaaccttgtgctgggtgtgctgtcaggggagtttgccaaagaaagggaacgggtggagaaccggcgggcttttctgaagctgaggcggcaacaacagattgaacgtgagctcaatgggtacatggagtggatctcaaaagcagaagaggtgatcctcgccgaggatgaaactgacggggagcagaggcatccctttgatggagctctgcggagaaccaccataaagaaaagcaagacagatttgctcaaccccgaagaggctgaggatcagctggctgatatagcctctgtgggttctcccttcgcccgagccagcattaaaagtgccaagctggagaactcgaccttttttcacaaaaaggagaggaggatgcgtttctacatccgccgcatggtcaaaactcaggccttctactggactgtactcagtttggtagctctcaacacgctgtgtgttgctattgttcactacaaccagcccgagtggctctccgacttcctttactatgcagaattcattttcttaggactctttatgtccgaaatgtttataaaaatgtacgggcttgggacgcggccttacttccactcttccttcaactgctttgactgtggggttatcattgggagcatcttcgaggtcatctgggctgtcataaaacctggcacatcctttggaatcagcgtgttacgagccctcaggttattgcgtattttcaaagtcacaaagtactgggcatctctcagaaacctggtcgtctctctcctcaactccatgaagtccatcatcagcctgttgtttctccttttcctgttcattgtcgtcttcgcccttttgggaatgcaactcttcggcggccagtttaatttcgatgaagggactcctcccaccaacttcgatacttttccagcagcaataatgacggtgtttcagatcctgacgggcgaagactggaacgaggtcatgtacgacgggatcaagtctcaggggggcgtgcagggcggcatggtgttctccatctatttcattgtactgacgctctttgggaactacaccctcctgaatgtgttcttggccatcgctgtggacaatctggccaacgcccaggagctcaccaaggtggaggcggacgagcaagaggaagaagaagcagcgaaccagaaacttgccctacagaaagccaaggaggtggcagaagtgagtcctctgtccgcggccaacatgtctatagctgtgaaagagcaacagaagaatcaaaagccagccaagtccgtgtgggagcagcggaccagtgagatgcgaaagcagaacttgctggccagccgggaggccctgtataacgaaatggacccggacgagcgctggaaggctgcctacacgcggcacctgcggccagacatgaagacgcacttggaccggccgctggtggtggacccgcaggagaaccgcaacaacaacaccaacaagagccgggcggccgagcccaccgtggaccagcgcctcggccagcagcgcgccgaggacttcctcaggaaacaggcccgctaccacgatcgggcccgggaccccagcggctcggcgggcctggacgcacggaggccctgggcgggaagccaggaggccgagctgagccgggagggaccctacggccgcgagtcggaccaccacgcccgggagggcagcctggagcaacccgggttctgggagggcgaggccgagcgaggcaaggccggggacccccaccggaggcacgtgcaccggcaggggggcagcagggagagccgcagcgggtccccgcgcacgggcgcggacggggagcatcgacgtcatcgcgcgcaccgcaggcccggggaggagggtccggaggacaaggcggagcggagggcgcggcaccgcgagggcagccggccggcccggggcggcgagggcgagggcgagggccccgacgggggcgagcgcaggagaaggcaccggcatggcgctccagccacgtacgagggggacgcgcggagggaggacaaggagcggaggcatcggaggaggaaagagaaccagggctccggggtccctgtgtcgggccccaacctgtcaaccacccggccaatccagcaggacctgggccgccaagacccacccctggcagaggatattgacaacatgaagaacaacaagctggccaccgcggagtcggccgctccccacggcagccttggccacgccggcctgccccagagcccagccaagatgggaaacagcaccgaccccggccccatgctggccatccctgccatggccaccaacccccagaacgccgccagccgccggacgcccaacaacccggggaacccatccaatcccggcccccccaagacccccgagaatagccttatcgtcaccaaccccagcggcacccagaccaattcagctaagactgccaggaaacccgaccacaccacagtggacatccccccagcctgcccaccccccctcaaccacaccgtcgtacaagtgaacaaaaacgccaacccagacccactgccaaaaaaagaggaagagaagaaggaggaggaggaagacgaccgtggggaagacggccctaagccaatgcctccctatagctccatgttcatcctgtccacgaccaacccccttcgccgcctgtgccattacatcctgaacctgcgctactttgagatgtgcatcctcatggtcattgccatgagcagcatcgccctggccgccgaggaccctgtgcagcccaacgcacctcggaacaacgtgctgcgatactttgactacgtttttacaggcgtctttacctttgagatggtgatcaagatgattgacctggggctcgtcctgcatcagggtgcctacttccgtgacctctggaatattctcgacttcatagtggtcagtggggccctggtagcctttgccttcactggcaatagcaaaggaaaagacatcaacacgattaaatccctccgagtcctccgggtgctacgacctcttaaaaccatcaagcggctgccaaagctcaaggctgtgtttgactgtgtggtgaactcacttaaaaacgtcttcaacatcctcatcgtctacatgctattcatgttcatcttcgccgtggtggctgtgcagctcttcaaggggaaattcttccactgcactgacgagtccaaagagtttgagaaagattgtcgaggcaaatacctcctctacgagaagaatgaggtgaaggcgcgagaccgggagtggaagaagtatgaattccattacgacaatgtgctgtgggctctgctgaccctcttcaccgtgtccacgggagaaggctggccacaggtcctcaagcattcggtggacgccacctttgagaaccagggccccagccccgggtaccgcatggagatgtccattttctacgtcgtctactttgtggtgttccccttcttctttgtcaatatctttgtggccttgatcatcatcaccttccaggagcaaggggacaagatgatggaggaatacagcctggagaaaaatgagagggcctgcattgatttcgccatcagcgccaagccgctgacccgacacatgccgcagaacaagcagagcttccagtaccgcatgtggcagttcgtggtgtctccgcctttcgagtacacgatcatggccatgatcgccctcaacaccatcgtgcttatgatgaagttctatggggcttctgttgcttatgaaaatgccctgcgggtgttcaacatcgtcttcacctccctcttctctctggaatgtgtgctgaaagtcatggcttttgggattctgaattatttccgcgatgcctggaacatcttcgactttgtgactgttctgggcagcatcaccgatatcctcgtgactgagtttgggaatccgaataacttcatcaacctgagctttctccgcctcttccgagctgcccggctcatcaaacttctccgtcagggttacaccatccgcattcttctctggacctttgtgcagtccttcaaggccctgccttatgtctgtctgctgatcgccatgctcttcttcatctatgccatcattgggatgcaggtgtttggtaacattggcatcgacgtggaggacgaggacagtgatgaagatgagttccaaatcactgagcacaataacttccggaccttcttccaggccctcatgcttctcttccggagtgccaccggggaagcttggcacaacatcatgctttcctgcctcagcgggaaaccgtgtgataagaactctggcatcctgactcgagagtgtggcaatgaatttgcttatttttactttgtttccttcatcttcctctgctcgtttctgatgctgaatctctttgtcgccgtcatcatggacaactttgagtacctcacccgagactcctccatcctgggcccccaccacctggatgagtacgtgcgtgtctgggccgagtatgaccccgcagcttggggccgcatgccttacctggacatgtatcagatgctgagacacatgtctccgcccctgggtctggggaagaagtgtccggccagagtggcttacaagcggcttctgcggatggacctgcccgtcgcagatgacaacaccgtccacttcaattccaccctcatggctctgatccgcacagccctggacatcaagattgccaagggaggagccgacaaacagcagatggacgctgagctgcggaaggagatgatggcgatttggcccaatctgtcccagaagacgctagacctgctggtcacacctcacaagtccacggacctcaccgtggggaagatctacgcagccatgatgatcatggagtactaccggcagagcaaggccaagaagctgcaggccatgcgcgaggagcaggaccggacacccctcatgttccagcgcatggagcccccgtccccaacgcaggaagggggacctggccagaacgccctcccctccacccagctggacccaggaggagccctgatggctcacgaaagcggcctcaaggagagcccgtcctgggtgacccagcgtgcccaggagatgttccagaagacgggcacatggagtccggaacaaggcccccctaccgacatgcccaacagccagcctaactctcagtccgtggagatgcgagagatgggcagagatggctactccgacagcgagcactacctccccatggaaggccagggccgggctgcctccatgccccgcctccctgcagagaaccagaggagaaggggccggccacgtgggaataacctcagtaccatctcagacaccagccccatgaagcgttcagcctccgtgctgggccccaaggcccgacgcctggacgattactcgctggagcgggtcccgcccgaggagaaccagcggcaccaccagcggcgccgcgaccgcagccaccgcgcctctgagcgctccctgggccgctacaccgatgtggacacaggcttggggacagacctgagcatgaccacccaatccggggacctgccgtcgaaggagcgggaccaggagcggggccggcccaaggatcggaagcatcgacagcaccaccaccaccaccaccaccaccaccatcccccgccccccgacaaggaccgctatgcccaggaacggccggaccacggccgggcacgggctcgggaccagcgctggtcccgctcgcccagcgagggccgagagcacatggcgcaccggcagtag

SEQ ID NO: 59 - DNA, 1992 bp, Homo Sapiens
atggccctgtgttatggaactttctggggctaccctaagatgctggaagctgccaatctcatggagggcctagtggatatcggcccttgggtcactcttcccagaggacagcctgaggtgttggagtggggcctcccaaaggatcaggactcagtggcctttgaggatgtggctgtgaacttcacccatgaggagtgggctttgctgggtccatcacagaagaatctctacagagatgtgatgcgagaaaccattaggaacctgaactgtataggaatgaaatgggaaaaccagaacattgatgatcagcaccaaaatctcaggagaaatccaaggtgtgatgtggtagagagatttggtaaaagtaaagatggtagtcagtgtggagaaaccttaagccagattcgaaatagtattgtaaacaagaacactcccgccagagtagatgcatgtggaagcagtgtgaatggagaagtcataatgggtcattcatccctgaattgctacatcagagttgatactggacacaaacaccgggagtgtcatgaatatgcagagaagtcatatacacataagcagtgtgggaaaggcttaagttatcgccactcctttcaaacatgtgaaaggcctcacactggaaagaaaccctatgattgtaaggaatgtggaaaaaccttcagttctcctggaaaccttcgaagacatatggtagtaaaaggtggagatggaccttataaatgtgaattgtgtgggaaagcctttttttggcccagtttattacgtatgcatgaaagaactcacactggagagaaaccatatgaatgtaagcagtgttctaaagccttccctgtttacagttcctatctaagacatgaaaaaatacacactggggagaaaccgtatgaatgtaagcagtgttctaaagccttccctgattacagttcatatctaagacatgaaagaactcacactggagagaaaccctacaaatgtaaacaatgtgggaaagccttcagtgtttccggttcccttcgagtacatgaaagaattcacactggagagaaaccctatacatgtaaacagtgtgggaaagcgttttgtcatcttggaagctttcaaagacacatgataatgcacagtggagatggacctcataaatgtaagatatgtgggaaaggctttgattttcctggttcagcacgaattcatgaaggaactcacactctagagaaaccctatgaatgtaagcaatgtgggaaattgttatctcatcgctcaagctttcgaagacacatgatggcacacactggagatggccctcataaatgcacagtatgtgggaaagcctttgattctcctagtgtatttcaaagacatgaaaggactcacactggagagaaaccctatgaatgcaagcaatgtgggaaagccttccgtacttccagttcccttcgaaaacatgaaacaacacacactggagagcaaccctataaatgtaaatgtggaaaagcttttagtgatttattttcctttcaaagtcatgaaacaacacacagtgaagaggagccttatgaatgtaaggagtgtgggaaagcatttagttcttttaaatacttttgtcgccatgaaaggactcacagtgaagaaaaatcttatgagtgtcaaatttgtggcaaagccttcagtcgtttcagttacttaaaaactcatgaaaggactcacacggcagagaagccatatgaatgtaagcaatgcaggaaagcattcttttggccctctttccttctaagacatgaaaggactcacactggagaaagaccctatgaatgtaaacactgtggtaaagccttcagtcgttccagtttctgtcgagaacatgaaagaactcacactggagagaagccctatgaatgtaaggaatgtgggaaagccttcagttctctcagttcctttaatagacataaaaggacacactggaaggatattctataa

SEQ ID NO: 60 - DNA, 4944 bp, Homo Sapiens
atgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgagagcagacactgcagcacgggcagcggcagtgccagcttcgcccacactgcccctccgccagcgggcgtcaaccccgacttggaggagccacctctaaaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactga

SEQ ID NO: 61 - DNA, 459 bp, Homo Sapiens
atgaggctgcccctcagccacagcccagagcacgtggagatggctttgctcagcaacatcctagcggcctattcctttgtctcagaaaatcctgagcgagcagctctgtactttgtttctggcgtgtgcatcgggctggtgctgaccctggctgctctggtgataaggatctcttgccacacagactgcaggcggcgtcccgggaagaagttcctgcaggacagagagagcagcagcgacagcagcgacagcgaggatggcagtgaggacaccgtgtccgatctctccgtgcggagacaccgccgcttcgagaggactttgaacaagaatgtgttcacctctgcggaggagctggagcgcgcccagcggctggaggagcgcgagcgcatcatcagggagatctggatgaatggccagcctgaggtgcccgggaccaggagcctgaatcgctactattag

SEQ ID NO: 62 - DNA, 1833 bp, Homo Sapiens
atggactcagtggcctttgaagatgtggctgtgaacttcacacaagaggagtgggctttgctgggtccatcacagaagagtctctacagaaatgtcatgcaggaaaccattaggaacctggactgtatagaaatgaaatgggaggaccagaacattggagatcagtgccaaaatgccaagagaaatctaagaagtcatacatgtgaaattaaagatgacagtcaatgtggagaaacttttggccagattccagatagtattgtgaacaagaacactcctcgagtaaatccatgtgacagtggtgagtgtggagaagtcgtcttgggtcattcgtctcttaattgcaacatcagagttgacactggacacaaatcatgtgagcatcaggaatatggagagaagccatatacacataaacaacgtgggaaagccatcagtcatcagcactccttccagacacatgaaaggccccccaccggaaagaaacccttcgattgtaaagaatgtgcaaaaacctttagttctcttggaaacctccgaagacacatggcggcacaccatggagatggaccttataaatgtaagttgtgtgggaaagcctttgtttggcccagtttatttcatttgcacgaaagaacacacactggagagaaaccgtatgaatgtaagcagtgttctaaagcctttcctttttacagttcctatctaagacatgaaagaatccacacgggagagaaagcgtatgaatgtaagcagtgttccaaagcctttcctgattacagtacctatctaagacatgagagaactcacaccggagagaaaccctataaatgtacacaatgtgggaaagccttcagctgttactattacactcgactacatgaaaggactcacacgggagaacaaccctatgcatgtaagcaatgtgggaaaacgttttatcatcacacaagctttcgaagacacatgataaggcacactggagacggaccacataaatgtaagatatgtgggaaaggctttgattgtcctagttcagttcgaaatcatgaaactactcacactggagagaaaccctatgaatgtaagcagtgtgggaaagtgttatctcatagctcgagctttcgaagtcacatgataacacacacaggagatggaccccagaaatgcaagatatgtgggaaagcctttggttgtcccagtttatttcaaagacatgaaaggactcacactggagagaaaccctatcaatgtaaacaatgtggtaaagccttcagtcttgccggttcccttcgaagacatgaagcaactcacactggagtgaaaccctataaatgtcagtgtgggaaagcctttagtgatctctcttcctttcaaaatcatgagacaactcacactggagagaagccatatgagtgtaaggaatgtgggaaagcattcagttgtttcaaatacctttctcaacataaaaggacccacacagtagaaaaaccttatgagtgtaaaacatgtagaaaagccttcagtcatttcagtaacttaaaagtccatgaaaggattcactctggagagaagccatatgaatgtaaggaatgtggaaaagcattctcttggctcacttgccttctacgacatgaaagaattcacactggagagaaaccctatgaatgtctacaatgtggtaaagccttcactcgttcccgtttccttcgaggacatgaaaaaactcacactggagagaagctgtatgaatgtaaggaatgtgggaaagcattgagttctctccgttccttgcatagacataaaaggactcactggaaagatactctctaa

SEQ ID NO: 63 - DNA, 1650 bp, Homo Sapiens
atgctggagaactacaagaatttggccacagtaggatatcagctcttcaaacccagtctgatctcttggctggaacaagaagagtctaggacagtgcagagaggtgatttccaagcttcagaatggaaagtgcaacttaaaaccaaagagttagcccttcagcaggatgttttgggggagccaacctccagtgggattcaaatgataggaagccacaacggaggggaggtcagtgatgttaagcaatgtggagatgtctccagtgaacactcatgccttaagacacatgtgagaactcaaaatagtgagaacacatttgagtgttatctgtatggagtagacttccttactctgcacaagaaaacctctactggagagcaacgttctgtatttagtcagtgtggaaaagccttcagcctgaacccagatgttgtttgccagagaacgtgcacaggagagaaagcttttgattgcagtgactctgggaaatccttcattaatcattcacaccttcagggacatttaagaactcacaatggagaaagtctccatgaatggaaggaatgtgggagaggctttattcactccacagaccttgctgtgcgtatacaaactcacaggtcagaaaaaccctacaaatgtaaggaatgtggaaaaggatttagatattctgcataccttaatattcacatgggaacccacactggagacaatccctatgagtgtaaggagtgtgggaaagccttcaccaggtcttgtcaacttactcagcacagaaaaactcacactggagagaaaccttataaatgtaaggattgtgggagagccttcactgtttcctcttgcttaagtcaacatatgaaaatccatgtgggtgagaagccttatgaatgcaaggaatgtgggatagccttcactagatcttctcaacttactgaacatttaaaaactcacactgcaaaggatccctttgaatgtaagatatgtggaaaatcctttagaaattcctcatgcctcagtgatcactttcgaattcacactggaataaaaccctataaatgtaaggattgtgggaaagccttcactcagaactcagaccttactaagcatgcacgaactcacagtggagagaggccctatgaatgtaaggaatgtggaaaggcctttgccagatcctctcgccttagtgaacatacaagaactcacactggagagaagccttttgaatgtgtcaaatgtgggaaagcctttgctatttcttcaaatcttagtggacatttgagaattcacactggagagaagccctttgagtgcctggaatgtggtaaagcatttacgcattcctccagtcttaataatcacatgcggacccacagcgccaaaaaaccattcacgtgtatggaatgtggcaaagcttttaagtttcccacgtgtgttaaccttcacatgcggatccacactggagaaaaaccctacaaatgtaaacagtgtgggaaatccttcagttactccaattcgtttcagttacatgaacgaactcacactggagagaaaccctatgaatgtaaggagtgcgggaaagccttcagttcttccagttcctttcgaaatcatgaaagaaggcatgcggatgagagactgtcagcataa

SEQ ID NO: 64 - DNA, 1926 bp, Homo Sapiens
atggactcagtggtctttgaggatgtggctgtgaacttcacccaggaggagtgggctttgctgggtccctctcagaagaaactctacagagatgtgatgcaagaaacctttgttaacttggcctctataggggaaaactgggaggagaagaacattgaagatcacaaaaatcaggggagaaagctaagaagtcatatggtagagaggctctgtgaaaggaaagaaggtagtcagtttggagaaaccatcagtcagactccaaatcctaaaccaaacaagaaaacttttactagagtaaaaccatatgaatgtagtgtgtgtggaaaggactatatgtgtcattcatctcttaataggcacatgagatctcatactgaacatagatcatatgaatatcacaaatatggagagaaatcatatgaatgtaaggaatgtgggaaaagattcagctttcgaagttcatttcgaatacatgaaagaactcacactggagagaaaccctataaatgtaaacagtgtggtaaggctttcagttggcccagttcctttcaaatacatgaaagaactcatactggagagaaaccttatgaatgtaaggaatgtgggaaggccttcatttatcacacaacctttcgaggacacatgagaatgcacacaggggagaaaccctataaatgtaaagaatgcgggaaaacgttcagtcatcccagttcttttcgaaatcatgaaagaactcactctggagagaaaccctatgaatgtaaacaatgtggaaaagctttcagatattaccaaacttttcaaatacatgaaaggactcacactggggaaaaaccctatcagtgtaagcaatgtggtaaagctcttagttgtcccacatcctttcgaagtcatgaaaggattcacactggagaaaaaccctataaatgtaaaaaatgtgggaaagccttcagttttcctagttcctttagaaaacatgaaagaattcatacaggagagaaaccctatgattgtaaggaatgtgggaaagcattcatttctcttccaagctatcgaagacatatgataatgcacactggaaatggaccttataaatgcaaggaatgtgggaaagcctttgattgtcctagttcttttcaaatccatgaacgaactcacactggagagaaaccctatgaatgtaaacagtgtggtaaagccttcagttgttccagttcctttcgaatgcatgaaagaactcacactggagagaaaccccatgaatgtaaacaatgtggtaaagccttcagttgttccagttctgttcgaatacatgaaaggactcacactggagagaaaccctatgaatgtaaacagtgtggtaaagccttcagttgttccagttcctttcgaatgcatgaaagaattcacactggagagaaaccctatgaatgtaaacagtgtggtaaagcctttagtttttctagttcctttcggatgcatgaaaggactcacactggagagaaaccctatgaatgtaaacaatgtggtaaagccttcagttgttccagttcctttcgaatgcatgaaaggactcacactggggagaaaccctatgaatgtaaacagtgtggtaaggcgtttagttgttccagttccattcgaatacatgaaaggactcacactggagagaaaccttatgagtgtaaacaatgtggtaaggccttcagttgttctagttctgttcgaatgcatgaaaggactcacactggagtgaaaccctatgaatgtaaacaatgtgacaaagccttcagttgctcacgttcctttcgaatccatgaacgaactcacactggagagaaaccctatgcatgtcaacaatgtggtaaagccttcaagtgttcccgttcctttcgaatacatgaaagagttcatagtggagagtaa

SEQ ID NO: 65 - DNA, 228 bp, Homo Sapiens
atgatcggagacatcctgctgttcgggacgttgctgatgaatgccggggcggtgctgaactttaagctgaaaaagaaggacacgcagggctttggggaggagtccagggagcccagcacaggtgacaacatccgggaattcttgctgagcctcagatactttcgaatcttcatcgccctgtggaacatcttcatgatgttctgcatgattgtgctgttcggctcttga

SEQ ID NO: 66 - DNA, 1515 bp, Homo Sapiens
atggaggccgccgcccagttcttcgtcgagagcccggacgtggtctacggccccgaggccatcgaggcgcaatacgagtaccggacgacgcgcgtcagccgcgagggtggcgttctcaaggaggccaactactacggctcgctgactcaggcgggcaccgtgagcctgggcctggacgccgagggccaggaggtgttcgtacccttcagcgcggtgctgcccatggtggcgcccaacgacctcgtgttcgatggctgggacatctcgtcgctgaacctggccgaggcgatgcggcgcgcgaaggtgctggactgggggctgcaggagcaactgtggccgcacatggaggccctgcggccccggccttctgtttacatccccgaattcatcgcggccaaccagagcgcgcgcgcggacaacctcatcccaggctcgcgtgcgcagcagctggagcagatccgcagggacatccgagacttccggtctagcgcggggctggacaaagtcatagtgctgtggacggcgaacacggagcgcttctgtgaggtgattccaggcctcaacgacacagccgagaacctgctgcgcaccattgagctcggtctggaggtgtcgccctccacgctcttcgccgtggccagcatcctggagggctgtgccttcctcaatgggtctccgcagaacaccctggtgcccggagctcttgagctcgcgtggcagcaccgggtttttgtgggcggagatgacttcaagtcaggccagaccaaagtcaagtccgtgcttgtggacttcctcattggctccggcctcaagaccatgtccatcgtgagttacaaccacctgggcaacaacgatggggagaacctatcggcgccattgcagttccgctctaaggaggtgtccaagagcaacgtggtggacgacatggtgcagagcaacccagtgctctatacgcccggcgaagagcctgaccactgcgtggtcatcaagtatgtgccgtacgtgggtgacagcaagcgcgcgctggatgagtatacctcggagctgatgctgggcggaaccaacacactggtgctgcacaacacgtgtgaggactcgctgctggccgcacccatcatgctggacctagcgctgctgaccgagctgtgccagcgcgtgagcttctgcactgacatggaccccgagccgcagaccttccaccccgtgctgtccctgctcagcttcctcttcaaggcgccactagtgccgcccggcagcccggtggtcaatgcgcttttccgccagcgcagctgcatcgagaacatcctcagggcctgcgtggggctcccgccacagaaccacatgctcctggaacacaaaatggagcgcccagggcccagcctcaagcgagttggacccgtggctgccacctaccctatgttgaacaagaaaggaccggtacccgctgccaccaatggctgcaccggtgatgccaatgggcatctgcaagaggagcccccaatgcccaccacctga

SEQ ID NO: 67 - DNA, 1929 bp, Homo Sapiens
atggactcagtctcctttgaggatgtggccgtgaacttcaccctggaggagtgggctttgctggattcttcacagaaaaagctctatgaagatgtgatgcaggagaccttcaaaaacctggtttgtctaggaaaaaagtgggaagaccaggacattgaagatgaccacagaaaccaggggaaaaatcgaagatgtcatatggttgagagactctgtgaaagtagaagaggtagcaaatgtggagaaaccactagccagatgccaaatgttaatatcaacaaggaaacttttactggagcaaaaccacatgaatgcagcttttgtggaagagacttcattcatcattcgtcccttaataggcacatgagatctcacactggacagaaaccaaatgagtatcaggaatatgaaaagcaaccatgtaaatgtaaagcagttgggaaaaccttcagttatcaccactgctttcgcaaacatgaaagaactcacactggagtgaagccctatgaatgtaaacagtgtgggaaagcctttatatattaccagccatttcaaagacatgaaaggactcatgctggacagaaaccctatgaatgtaagcaatgtggaaaaacctttatatattaccagtcttttcaaaaacatgctcatactggaaagaaaccctatgaatgtaaacagtgtgggaaagcctttatatgttaccaatcttttcaaagacacaaaaggactcacactggagagaaaccctatgaatgtaagcaatgtggtaaggctttcagttgtcccacatactttcgaactcatgaaagaactcacactggagaaaaaccctacaaatgtaaagaatgtggtaaagccttcagttttctcagttcttttcgaaggcataaaaggactcatagtggagagaaaccctatgaatgtaaagaatgtggaaaagccttcttttattctgcaagctttcgagcacatgtaataatacacactggggctcgaccttataaatgtaaagaatgtgggaaagccttcaactcttctaattcctgtcgagtgcatgaaagaactcatattggagaaaaaccatatgaatgtaaacgatgtggcaaatcattcagttggtccatttctcttcgattgcatgaaagaactcatactggagagaaaccttatgagtgtaaacagtgtcataaaaccttcagtttttcaagttcccttcgagaacacgaaacaactcacactggagagaaaccctatgaatgtaaacaatgtggtaaaaccttcagtttttcaagttcccttcaaagacatgaaaggactcacaatgcagagaaaccctatgaatgtaaacagtgtgggaaagccttcaggtgttcaagttattttcgaattcatgaaaggtcacacactggagagaaaccctatgaatgtaaacagtgtggaaaagttttcattcgttccagttcctttcgactgcatgaaagaacacacactggagagaaaccctatgaatgtaaactatgcggtaaaaccttcagtttttcaagttcccttcgagaacatgaaaaaattcacactggaaataagccttttgagtgtaagcaatgtggtaaggccttccttcgttccagtcaaattcgattgcatgaaaggactcacactggagagaaaccgtatcaatgtaaacaatgtggaaaagccttcatttcttccagtaaatttcgaatgcatgagagaactcacacgggagagaaaccctatcgatgtaaacaatgtgggaaagccttcagattttcaagttctgttcgaattcatgaaaggtctcacactggagagaaaccttatgaatgcaaacaatgtggaaaagccttcatttcttccagtcactttcgactgcatgaaaggactcatatgggagagaaagtctaa

SEQ ID NO: 68 - DNA, 1863 bp, Homo Sapiens
atgggaccattgcaatttagagatgtggccatagaattctctctggaggagtggcattgcctggacactgcacagcggaatctatataggaatgtgatgttagagaactacagtaacctggtcttccttggtattgttgtctctaagccagacctgatcgcccatctggagcaaggaaaaaaacctttgactatgaagagacatgagatggtagccaacccctcagttatatgttctcattttgcccaagatctttggccagagcagaacataaaagattctttccaaaaagtgatactgagaagatatgaaaaacgtggacatggaaatttacagttaataaaaaggtgtgaaagtgtagatgagtgtaaggtgcacacaggaggttataatggacttaaccagtgtagtacaactacccagagcaaagtatttcaatgtgataaatatgggaaagtctttcataaattttcaaattcaaatagacataatataagacatactgaaaaaaaacctttcaaatgcatagaatgtggcaaagcttttaaccagttctcaacccttataacacataagaaaattcatactggagagaaaccctacatttgtgaagaatgtggcaaagcctttaagtactcctctgcccttaatacacataagagaattcatactggagagaaaccatacaagtgtgataaatgtgacaaagcctttattgcatcctcaacccttagtaaacatgagatcattcatactggaaagaaaccctacaagtgtgaagaatgtggcaaagcttttaaccaatcctcgacacttactaaacataagaaaattcatactggagagaaaccctacaaatgtgaagaatgtggcaaagcttttaaccaatcctcaacacttactaaacataagaaaattcatactggagagaagccctacgtttgtgaagaatgtggcaaagcctttaagtactcccgtatccttactacacataagagaattcatactggagagaaaccatacaagtgtaataaatgtggcaaagcctttattgcatcctcaacccttagtagacatgagttcattcatatgggaaagaaacattacaaatgtgaagaatgtggcaaagccttcatttggtcctcagtcctaactagacataagagagttcatactggagagaagccctacaaatgtgaagaatgtggcaaagcctttaagtactcctctacccttagttcacataagagaagtcatactggagagaaaccctacaaatgtgaagaatgtggcaaagcctttgttgcatcctcaacccttagtaaacatgagatcattcatactggaaagaaaccctacaagtgtgaagaatgtggcaaagcttttaaccagtcctcatcccttactaaacataagaaaattcatactggagagaaaccctacaaatgtgaagaatgtggcaaagcttttaaccagtcctcttcccttactaaacataagaaaattcatactggagagaaaccctacaaatgtgaagaatgtggcaaagcttttaaccagtcctcaacccttattaaacataagaaaattcatactagagagaaaccctacaaatgtgaagaatgtggcaaagcttttcacctatccacacaccttactacacataagatacttcatactggagagaaaccttatagatgtagagaatgtggcaaagcttttaaccattctgcaaccctttcttcacataagaaaatccattctggagagaaaccatacgagtgtgataaatgtggcaaagcctttatttcaccctcaagccttagtagacatgagataattcatactggggagaaaccctag

SEQ ID NO: 69 - DNA, 1500 bp, Homo Sapiens
atgggaccattgcaatttagagatgtggccatagaattctctctggaggagtggcattgcctggacactgcacagcggaatttatatagggatgtgatgttagagaactacagaaacttggtcttccttggtattgttgtctctaagccagacctggttacctgtctggagcaaggaaaaaaacctttaactatggaaagacatgagatgattgccaaacccccagttatgagttctcattttgcccaagacctttggccagagaacatacaaaattctttccaaatagggatgctgagaagatatgaagaatgcagacatgacaatttacagttaaaaaaaggctgtaaaagcgtgggtgagcataaggtgcacaaaggaggttataatggacttaaccaatgtttgacaactacccagaaagaaatatttcaatgtgataaatatggaaaagtctttcataagttttcaaattcaaacacatataagacaagacatactggaataaatcttttcaaatgtataatatgtggcaaagcttttaaacggtcctcaacccttactacacataagaaaattcatactggagagaaaccttacagatgtgaagaatgtggcaaagcttttaaccaatctgcaaaccttactacacataagagaattcataccggagagaaaccctacagatgtgaagaatgtggcaaagcctttaagcagtcctcaaaccttactacacataagaaaattcatactggagagaaaccctacaaatgtgaagaatgtggcaaagccttcaaccgatccacagaccttactacacataagatagttcatactggagagaaaccctacaaatgtgaagaatgtggcaaagcctttaagcacccctcacacgttaccacacataagaaaattcatactagagggaaaccctacaactgtgaagaatgtggcaaatcctttaagcactgctctaaccttactatacataagagaattcatacaggagagaaaccctacaaatgtgaagaatgtggcaaagcctttcacctatcctcacaccttactacacataagatacttcatactggagagaaaccctacagatgtagagaatgtggcaaagcttttaaccattccacaacccttttttcacatgagaaaattcatactggagagaaaccctacaaatgtgatgaatgtggcaaaacctttacctggccctcaatcctctccaaacataaaagaactcatactggagagaaaccctacaaatgtgaagaatgtggcaaatcctttactgcatcctcaactctaactacacataagagaattcatactggagagaaaccttacaaatgtgaagaatgtggcaaagcttttaactggtcctcagaccttaataaacataagaaaattcatattgaacgaaaaccctacatagtgaagaatgtgacagatcttttaaatgttcctccacttttaattagcataagataa

SEQ ID NO: 70 - DNA, 1482 bp, Homo Sapiens
atgttgaaagcccttttcctaactatgctgactctggcgctggtcaagtcacaggacaccgaagaaaccatcacgtacacgcaatgcactgacggatatgagtgggatcctgtgagacagcaatgcaaagatattgatgaatgtgacattgtcccagacgcttgtaaaggtggaatgaagtgtgtcaaccactatggaggatacctctgccttccgaaaacagcccagattattgtcaataatgaacagcctcagcaggaaacacaaccagcagaaggaacctcaggggcaaccaccggggttgtagctgccagcagcatggcaaccagtggagtgttgcccgggggtggttttgtggccagtgctgctgcagtcgcaggccctgaaatgcagactggccgaaataactttgtcatccggcggaacccagctgaccctcagcgcattccctccaacccttcccaccgtatccagtgtgcagcaggctacgagcaaagtgaacacaacgtgtgccaagacatagacgagtgcactgcagggacgcacaactgtagagcagaccaagtgtgcatcaatttacggggatcctttgcatgtcagtgccctcctggatatcagaagcgaggggagcagtgcgtagacatagatgaatgtaccatccctccatattgccaccaaagatgcgtgaatacaccaggctcattttattgccagtgcagtcctgggtttcaattggcagcaaacaactatacctgcgtagatataaatgaatgtgatgccagcaatcaatgtgctcagcagtgctacaacattcttggttcattcatctgtcagtgcaatcaaggatatgagctaagcagtgacaggctcaactgtgaagacattgatgaatgcagaacctcaagctacctgtgtcaatatcaatgtgtcaatgaacctgggaaattctcatgtatgtgcccccagggataccaagtggtgagaagtagaacatgtcaagatataaatgagtgtgagaccacaaatgaatgccgggaggatgaaatgtgttggaattatcatggcggcttccgttgttatccacgaaatccttgtcaagatccctacattctaacaccagagaaccgatgtgtttgcccagtctcaaatgccatgtgccgagaactgccccagtcaatagtctacaaatacatgagcatccgatctgataggtctgtgccatcagacatcttccagatacaggccacaactatttatgccaacaccatcaatacttttcggattaaatctggaaatgaaaatggagagttctacctacgacaaacaagtcctgtaagtgcaatgcttgtgctcgtgaagtcattatcaggaccaagagaacatatcgtggacctggagatgctgacagtcagcagtatagggaccttccgcacaagctctgtgttaagattgacaataatagtggggccattttcattttag

(19) United States
(12) Patent Application Publication
(10) Pub. No.: US 2013/0316923 A1
(43) Pub. Date: Nov. 28, 2013

(54) METHODS FOR PREDICTING ANTI-INTEGRIN ANTIBODY RESPONSE

(71) Applicant: Janssen Biotech, Inc., (US)

(72) Inventors: Huiqing Liu, Spring House, PA (US); Jaehong Park, Spring House, PA (US)

(73) Assignee: JANSSEN BIOTECH, INC., Horsham, PA (US)

(21) Appl. No.: 13/804,069

(22) Filed: Mar. 14, 2013

Related U.S. Application Data
(60) Provisional application No. 61/642,486, filed on May 4, 2012.

(51) Int. Cl. C12Q 1/68

(52) U.S. Cl. CPC C12Q 1/6886
USPC 506/9

(57) ABSTRACT

The present invention relates to methods and procedures for predicting responsiveness to anti-integrin αv monoclonal antibody.

### PRIOR APPLICATION

This application claims priority to U.S. Application No. 61/642,486, filed May 4, 2012, which is entirely incorporated herein by reference.

### BACKGROUND OF THE INVENTION

1. Field of the Invention

The present invention relates to methods and procedures for predicting responsiveness to anti-integrin αv monoclonal antibody.

2. Background of the Invention

Non-small cell lung cancer (NSCLC) has generally poor prognosis and the response rate to chemotherapy or targeted therapy is low. Recent clinical trials suggest that adjuvant chemotherapy against microscopic metastatic disease improves the survival of resected NSCLC patients. The 5-year survival rate (overall and progression-free survival) has shown a modest 4-15% improvement, unfortunately with serious adverse effects. Lack of understanding of the tumor heterogeneity at molecular level is considered the major reason for the poor prognosis and poor response rate.

Rapid advancement in genetic and genomic technologies has resulted in better understanding of the molecular characters of tumors at individual patient level, making personalized medicine an effective and powerful new weapon against cancer. For example, expression-profile based multiple-gene diagnosis or prognosis signatures have been developed for breast cancer and lung cancer. Companion diagnosis is another area where genetic and genomic technology has made personalized medicine a possibility. In lung cancer, mutations in EGFR and K-RAS strongly predicted the efficacy of EGFR antagonist therapy. In a prospective study of customized trial of selective treatment with Tarceva® (erlotinib), an EGFR tyrosine kinase inhibitor, an overall response rate was higher than 70% in the targeted EGFR mutant population in multiple studies. Independently, K-RAS mutation status has been shown as a biomarker of resistance against tyrosine kinase inhibitors ((IRESSA™ (gefitinib) and Tarceva® (erlotinib)) in lung cancer.

Intetumumab (CNTO 95) is a fully human monoclonal antibody (mAb) that inhibits all five types of αᵥ integrins including αᵥβ₁, αᵥβ₅, αᵥβ₆, and αᵥβ₈. Previous studies have shown that Intetumumab exhibits both anti-tumor and anti-angiogenic activities. In a Phase I clinical study, Intetumumab was shown to be generally safe and well tolerated.

In general, the effectiveness of treatment and clinical study design is impacted by the availablility of markers predicting the patient population who will respond to treatment. Thus, there is a need for identification of markers facilitating patient stratification strategies for effective treatment and clinical study designs.

### BRIEF DESCRIPTION OF THE DRAWINGS

FIG. 1. The effect of Intetumumab on cell proliferation/viability in lung cancer cell lines.

FIG. 2. Flowchart of data and analysis for signature identification of Intetumumab response from human lung cancer cell lines.

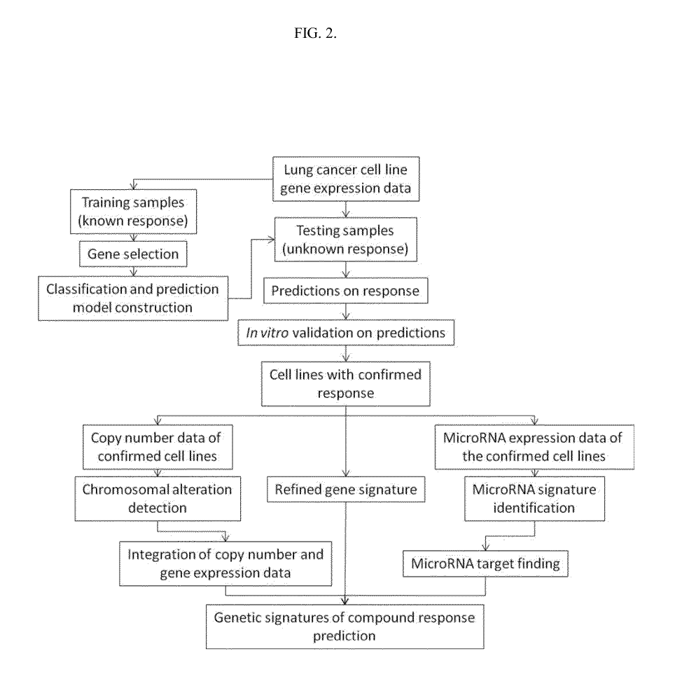

FIG. 3. Chromosomal regions that are amplified or deleted in at least 7 out of 8 resistant cell lines but no change in at least 4 out of 5 sensitive cell lines.

FIG. 4. Expression of epithelial to mesenchymal transition (EMT) markers among tested lung cancer cell lines. A) Heat map of expression patterns for EMT and tumor metastasis-related microRNAs and genes. Data is normalized and the correlation on the right end is to hsa-miR-200c except TWIST1 is to hsa-miR-10b. B) A plot showing strong inverse correlation between the expression of ZEB1 and miR-200c.

### SUMMARY OF THE INVENTION

One aspect of the invention is a method of identifying a subject with cancer who is most likely to benefit from treatment with anti-integrin antibody Intetumumab, comprising obtaining a sample of nucleic acids from a specimen obtained from a subject with cancer; determining expression levels of nucleic acids hybridizing with panels of probes having sequences of certain SEQ ID NOs: or fragments thereof; calculating a prediction score (Score) for the first panel of probes, wherein the prediction score is defined as

$$Score = {\sum }_{i = 1}^{5} {a}_{i} {p}_{i} {s}_{i}$$

where for each classification model i (i=1,2,3,4,5), aᵢ is its leave-one-out cross validation (LOOCV) accuracy, pᵢ is its prediction for the sample with 1 for sensitive and −1 for resistant, sᵢ is a switch between 0 and 1 and is set to 1 when aᵢ>=87.5%; otherwise, 0; and identifying the subject as one most likely to benefit from treatment with the anti-integrin antibody Intetumumab when the calculated prediction score is over zero (>0).

### DETAILED DESCRIPTION OF THE INVENTION

### Definitions

A “biomarker” is defined as ‘a characteristic that is objectively measured and evaluated as an objective indicator of normal biological processes, pathogenic processes, or pharmacologic responses to a therapeutic intervention’ by the Biomarkers Definitions Working Group (Atkinson et al. 2001 Clin Pharm Therap 69(3):89-95). Thus, an anatomic or physiologic process can serve as a biomarker, for example, range of motion, as can levels of proteins, gene expression (mRNA), small molecules, metabolites or minerals, provided there is a validated link between the biomarker and a relevant physiologic, toxicologic, pharmacologic, or clinical outcome.

By “sample” or “patient's sample” is meant a specimen which is a cell, tissue, or fluid or portion thereof extracted, produced, collected, or otherwise obtained from a patient suspected to having or having presented with symptoms associated with cancer. An exemplary sample is a DNA or RNA sample isolated from patient's cell or tissue.

By “sensitive” or “responsive” is meant that the proliferation of a cell line is reduced by about at least 20% in response to Intetumumab administered into culture media at a concentration of 20 μg/ml when compared to the same cell line grown without the presence of Intetumumab. Typically, cell line is a lung cancer cell line cultured on vitronectin—coated plates.

By “resistant” is meant that the proliferation of a cell line is reduced a maximum of about 5% in response to Intetumumab administered into culture media at a concentration of 20 μg/ml when compared to the same cell line grown without the presence of Intetumumab. Typically, cell line is a lung cancer cell line cultured on vitronectin—coated plates.

A “decreased level” or “lower level” of a biomarker refers to a level that is quantifiably less than a predetermined value which may be a control value, e.g., the value found in normal subjects, or may also called the “cutoff value” and above the lower limit of quantitation (LLOQ). This determined “cutoff value” is specific for the algorithm and parameters related to patient sampling and treatment conditions.

A “higher level” or “elevated level” of a biomarker refers to a level that is quantifiably elevated relative to a predetermined value, which may be a control value, e.g., the value found in normal subjects or may also be called the “cutoff value.” This “cutoff value” is specific for the algorithm and parameters related to patient sampling and treatment conditions.

The terms “array” or “microarray” or “biochip” or “chip” as used herein refer to articles of manufacture or devices comprising a plurality of immobilized target elements, each target element comprising a “clone,” “feature,” “spot” or defined area comprising a particular composition, such as a biological molecule, e.g., a nucleic acid molecule or polypeptide, immobilized to a solid surface.

“Complement of” or “complementary to” a nucleic acid sequence of the invention refers to a polynucleotide molecule having a complementary base sequence and reverse orientation as compared to a first polynucleotide.

A “Nucleic acid” as used herein refers to a deoxyribonucleotide (DNA) or ribonucleotide (RNA) in either single- or double-stranded form. The term encompasses nucleic acids containing known analogues of natural nucleotides. The term nucleic acid is used interchangeably with gene, DNA, RNA, cDNA, mRNA, oligonucleotide primer, probe and amplification product.

The present invention relates to a method of identifying patient or cell populations that are responsive or resistant to Intetumumab treatment; and therefore patients suitable for treatment with intetumumab. The present invention provides panels of differentially expressed gene sets that discriminate Intetumumab resistant and sensitive cell lines and/or patients responsive or non-responsive for Intetumumab treatment.

Methods of isolating polynucleotides from various samples such as tissues or cells as well as hybridization methods, expression profiling, and methods of making oligonucleotide arrays are well known in the art.

### Use of Reference/Training Datasets to Determine Parameters of Analytical Process

Using any suitable learning algorithm, an appropriate reference or training dataset is used to determine the parameters of the process to be used for classification, i.e., develop a predictive model.

The reference, or training dataset, to be used will depend on the desired classification to be determined, e.g., resistant or sensitive. The dataset may include data from one or two classes.

For example, to use a supervised learning algorithm to determine the parameters for an analytic process used to predict response to lung cancer therapy agent, a dataset comprising known resistant or sensitive samples are used as a training set.

### Statistical Analysis

The following are examples of the types of statistical analysis methods that are available to one of skill in the art to aid in the practice of the disclosed methods. These and other statistical methods may be used to identify subsets of the markers and other indicia that will form a dataset to be used. In addition, these and other statistical methods may be used to generate the process that will be used with the dataset to generate the result. Biomarkers and their corresponding features (e.g., expression levels or serum levels) are used to develop a process, or plurality of processes, that discriminate between classes of patients or cell lines, e.g., those who will respond to the treatment and those who are resistant to the treatment. Once a process has been built using these exemplary data analysis algorithms or other techniques known in the art, the process can be used to classify a test subject into one of the two or more phenotypic classes (e.g., a patient or cell line predicted to respond to the treatment or patient or cell line predicted not to response to the treatment). This is accomplished by applying the process to a marker profile obtained from the test subject. Such processes, therefore, have value as diagnostic indicators.

Thus, in some embodiments, the result in the above-described binary decision situation has four possible outcomes: (i) a true responder, where the process indicates that the subject will be a responder to therapy and the subject responds to therapy during the definite time period (true positive, TP); (ii) false responder, where the process indicates that the subject will be a responder to therapy and the subject does not respond to therapy during the definite time period (false positive, FP); (iii) true non-responder, where the process indicates that the subject will not be a responder to therapy and the subject does not respond to therapy during the definite time period (true negative, TN); or (iv) false non-responder, where the process indicates that the patient will not be a responder to therapy and the subject does in fact respond to therapy during the definite time period (false negative, FN).

Relevant data analysis algorithms for developing a process include, but are not limited to, discriminant analysis including linear, logistic, and more flexible discrimination techniques (see, e.g., Gnanadesikan, 1977, Methods for Statistical Data Analysis of Multivariate Observations, New York: Wiley 1977, which is hereby incorporated by reference herein in its entirety); tree-based algorithms such as classification and regression trees (CART) and variants (see, e.g., Breiman, 1984, Classification and Regression Trees, Belmont, Calif.; Wadsworth International Group); generalized additive models (see, e.g., Tibshirani, 1990, Generalized Additive Models, London: Chapman and Hall); and neural networks (see, e.g., Neal, 1996, Bayesian Learning for Neural Networks, New York: Springer-Verlag; and Insua, 1998); Feedforward neural networks for nonparametric regression In: Practical Nonparametric and Semiparametric Bayesian Statistics, pp. 181-194, New York: Springer. These references are hereby incorporated by reference in their entirety.

While such algorithms may be used to construct a process and/or increase the speed and efficiency of the application of the process and to avoid investigator bias, one of ordinary skill in the art will realize that a computer-based device is not required to carry out the methods of using the classification models of the present invention.

An exemplary algorithm to generate the process to discriminate between classes of patients or cell lines is a combination of three classification methods provided by ArrayStudio, k-Nearest Neighbor (k-NN), Linear Discriminant Analysis (LDA) and Support Vector Machine (SVM). For k-NN, k=1 or 3 can be selected while for SVM, cost=0 and Gamma=2⁻⁴ or 2⁻³ can be set for radial basis function kernel. As a result, five models are generated for each classification task: 1-NN, 3-NN, LDA, and two SVMs. Model evaluation and discrimination between classes of patients or cell lines is based on the accuracy of leave-one-out cross validation (LOOCV) on the training samples. Prediction to assess weather patient or cell line is sensitive or resistant to treatment is made by the combination of the prediction from the individual models whose LOOCV accuracy>=87.5% (i.e. no or one mistake among the 8 training samples). In detail, a “prediction score”, “Score”, as used herein for a testing sample is defined as

$$Score = {\sum }_{i = 1}^{5} {a}_{i} {p}_{i} {s}_{i}$$

Where for each classification model described above i (i=1,2,3,4,5), aᵢ is its LOOCV accuracy, pᵢ is its prediction for the sample with 1 for sensitive and −1 for resistant, sᵢ is a switch between 0 and 1 and is set to 1 when aᵢ>=87.5%; otherwise, 0. The final response prediction for the patient or cell line is Sensitive if Score>0 or Resistant if Score<0; otherwise, unknown

### Marker Sets for Identification Responders and Non-Responders

Analyses was focused on defining those marker sets that can be used to distinguish a cancer patient or cell line responding to Intetumumab treatment and a cancer patient or a cell line resistant to the treatment.

In one embodiment, the gene marker set is a set of Affymetrix probes or a set of genes or fragments thereof shown in Table 1 (“Set 1”). A particular probe set ID represents a fragment of a corresponding gene.

TABLE 1

| | SEQ ID | Corresponding | SEQ ID |
|---|---|---|---|
| Probe Set ID | NO: | Gene | NO: |
| 224463_s_at | 1 | C11orf70 | 11 |
| 241198_s_at | 2 | C11orf70 | 11 |
| 230747_s_at | 3 | TTC39C | 12 |
| 218147_s_at | 4 | GLT8D1 | 13 |
| 205780_at | 5 | BIK | 14 |
| 223805_at | 6 | OSBPL6 | 15 |
| 238856_s_at | 7 | PANK2 | 16 |
| 232202_at | 8 | — | n/a |
| 204678_s_at | 9 | KCNK1 | 17 |
| 239217_x_at | 10 | ABCC3 | 18 |

In one embodiment, the gene marker set is a set of probes or a set of genes or fragments thereof shown in Table 2 (“Set 2”).

TABLE 2

| | SEQ ID | Corresponding | SEQ ID |
|---|---|---|---|
| Probe Set ID | NO: | Gene | NO: |
| 201387_s_at | 19 | UCHL1 | 29 |
| 1567912_s_at | 20 | CT45-4 | 30 |
| 225710_at | 21 | GNB4 | 31 |
| 206858_s_at | 22 | HOXC4 /// HOXC6 | 32 |
| 209118_s_at | 23 | TUBA1A | 33 |
| 231736_x_at | 24 | MGST1 | 34 |
| 1565162_s_at | 25 | MGST1 | 35 |
| 33323_r_at | 26 | SFN | 36 |
| 201131_s_at | 27 | CDH1 | 37 |
| 224650_at | 28 | MAL2 | 38 |

In one embodiment, the gene marker set is a set of probes or a set of genes or fragments thereof shown in Table 3 (“Set 3”).

TABLE 3

| | SEQ ID | Corresponding | SEQ ID |
|---|---|---|---|
| Probe Set ID | NO: | Gene | NO: |
| 203718_at | 39 | PNPLA6 | 49 |
| 37986_at | 40 | EPOR | 50 |
| 209963_s_at | 41 | EPOR | 50 |
| 209962_at | 42 | EPOR | 50 |
| 242915_at | 43 | ZNF682 | 51 |
| 244552_at | 44 | ZNF788 | |
| 202927_at | 45 | PIN1 | 53 |
| 223024_at | 46 | AP1M1 | 54 |
| 212512_s_at | 47 | CARM1 | 55 |
| 223318_s_at | 48 | ALKBH7 | 56 |

In one embodiment, the gene marker set is a set of probes or a set of genes or fragments thereof shown in Table 4 (“Set 4”).

TABLE 4

| | | SEQ |
|---|---|---|
| Gene Symbol | Gene name | ID NO: |
| MARCH2 | membrane-associated ring finger (C3HC4) 2 | 57 |
| CACNA1A | calcium channel, voltage-dependent, | 58 |
| | P/Q type, alpha 1A subunit | |
| ZNF44 | Zinc finger protein 44 | 59 |
| SMARCA4 | SWI/SNF related, matrix associated, actin | 60 |
| | dependent regulator of chromatin, subf | |
| LOC147727 | hypothetical LOC147727 | |
| ZNF823 | zinc finger protein 823 | 62 |
| ZNF266 | zinc finger protein 266 | 63 |
| ZNF788 | zinc finger family member 788 | |
| ZNF709 | zinc finger protein 709 | 64 |
| C19orf42 | chromosome 19 open reading frame 42 | 65 |
| ISYNA1 | myo-inositol 1-phosphate synthase A1 | 66 |
| ZNF14 | zinc finger protein 14 | 67 |
| ZNF93 | zinc finger protein 93 | 68 |
| ZNF253 | zinc finger protein 253 | 69 |
| ZNF682 | zinc finger protein 682 | 51 |
| EFEMP1 | EGF-containing fibulin-like extracellular matrix | 70 |
| | protein 1 | |
| CYP26B1 | cytochrome P450, family 26, subfamily B, | 52 |
| | polypeptide 1 | |
| FAM176A | family with sequence similarity 176, member A | 61 |

It will be clear that the invention can be practiced otherwise than as particularly described in the foregoing description and examples. Numerous modifications and variations of the present invention are possible in light of the above teachings and, therefore, are within the scope of the appended claims.

### EXAMPLE 1

### Methods and Materials

### Cell Lines and Cell Proliferation Assay

Total 23 lung cancer cell lines from (ATCC, Manassas, Va.) or internal sources were used in the study (Table 5). All cells were maintained in RPMI-1640 media supplemented with 10% FBS, 1x non-essential amino acids, and sodium pyruvate. Cells were grown at 37° C. in the presence of 5% CO₂. A 96-well tissue culture plates were coated with 1 μg/ml (100 μL/well) of vitronectin overnight at 4° C. The following day, vitronectin was removed and plates were blocked by overnight with 1% bovine serum albumin (BSA) in phosphate-buffered saline (PBS) at 4° C. Prior to seeding cells, plates were washed with Dulbecco's PBS. Cells were plated at 5000 cells/well in 100 μL and were allowed to adhere overnight. The culture medium was then removed and serial dilutions of Intetumumab or PBS were added to appropriate wells in RPMI-1640 medium containing 2% FBS.

Plates were incubated for 72 hours and 20 μL of CellTitre 96 Aqueous One Solution reagent was added into each well of the 96-well assay plate containing the samples, Intetumumab or control in 100 μL of media. Plates were further incubated for 2 hours at 37° C. Absorbance was read at 490 nm.

For all cell lines, 3 replicates were assayed for each dose and treatment combinations. All replicates were used in the data analysis. Comparisons were computed in percentage relative growth to PBS control. Cells were called responsive, or sensitive, when the percentage relative growth was found below 80%.

### Gene Expression Profiling and Data Analysis

Global gene expression profiling of the 23 lung cancer cell lines was generated on Affymetrix HG-U133_Plus2 platform according to the manufacture's protocol (Affymetrix, Santa Clara, Calif.).

Three approaches were chosen to identify genes having significant expression change between resistant and sensitive cell lines.

The first approach used is a feature filtering approach “Informative/Non-Informative calls” (I/NI). I/NI perfoms repeated measures for each target transcript being represented by 11-20 different primer probes to assess the signal-to-noise ratio of the corresponding probe set in Affymetrix chips. The method has been implemented in R and can be downloaded from http://_www_bioinf_jku_at/software/_farms/_farms_html. In this study, RMA (Robust Multichip Average) algorithm was selected to normalize data.

The second approached used is USE-Fold (Uniform Significance of Expression Fold change) function in Genes@Work (http://_domino_watson_ibm_com/_comm/_research_projects_nsf/_pages/_gaw_index_htm 1). Different from I/NI, USE-Fold is a supervised procedure to distinguish if a change of gene

TABLE 5

| | | | Copy | |
|---|---|---|---|---|
| | | Gene | Number | MicroRNA |
| | | Expression | Data | Data |
| Cell Line | Description | Set | Available | Available |
| NCI-H1299 | non-small cell | Training | Y | Y |
| | lung cancer | | | |
| NCI-H1703 | lung | Training | Y | Y |
| | adenocarcinoma | | | |
| NCI-H522 | non-small cell | Training | N | Y |
| | lung cancer | | | |
| NCI-H1975 | non-small cell | Training | Y | Y |
| | lung cancer | | | |
| NCI-H1373 | lung | Training | Y | N |
| | adenocarcinoma | | | |
| NCI-H1944 | non-small cell | Training | N | N |
| | lung cancer | | | |
| NCI-H322 | lung carcinoma | Training | Y | N |
| NCI-H441 | lung | Training | Y | Y |
| | adenocarcinoma | | | |
| NCI-H1155 | non-small cell | Validation | Y | Y |
| | lung cancer | | | |
| NCI-H1581 | non-small cell | Validation | N | N |
| | lung cancer | | | |
| NCI-H2106 | non-small cell | Validation | Y | N |
| | lung cancer | | | |
| NCI-H226 | squamous cell | Validation | N | Y |
| | carcinoma | | | |
| NCI-H510A | small cell lung | Validation | N | Y |
| | cancer | | | |
| A549 | lung carcinoma | Validation | Y | Y |
| NCI-H1355 | lung | Validation | N | Y |
| | adenocarcinoma | | | |
| NCI-H1395 | lung | Validation | N | Y |
| | adenocarcinoma | | | |
| NCI-H1650 | lung | Validation | Y | Y |
| | adenocarcinoma | | | |
| NCI-H2122 | non-small cell | Validation | Y | Y |
| | lung cancer | | | |
| NCI-H2126 | non-small cell | Validation | N | Y |
| | lung cancer | | | |
| NCI-H2170 | squamous cell | Validation | N | Y |
| | carcinoma | | | |
| NCI-H23 | non-small cell | Validation | Y | Y |
| | lung cancer | | | |
| NCI-H358 | non-small cell | Validation | N | Y |
| | lung cancer | | | |
| NCI-H460 | large cell lung | Validation | Y | Y |
| | carcinoma | | | |

expression from one phenotype to another is purely from experimental noise. The algorithm models the experimental noise from replicated experiments within which the gene expression level changes can be only explained by experimental noise. If replicated experiments are not available, a default noise distribution model based on sample preparation and hybridization noise exclusive for Affymetrix microarrays will be used. Once the noise model is established, USE-Fold outputs significant genes based on a user defined confidence level (p-value).

The third approached was a fold change calculation and t-test conducted in Array Studio (http://_www_omicsoft_com/).

### Classification/Prediction Model Construction

Three classification methods provided by ArrayStudio were used in this study, k-Nearest Neighbor (k-NN), Linear Discriminant Analysis (LDA) and Support Vector Machine (SVM). For k-NN, k=1 or 3 was selected while for SVM, cost=0 and Gamma=2⁻⁴ or 2⁻³ were set for radial basis function kernel. Therefore, five models were generated for each classification task: 1-NN, 3-NN, LDA, and two SVMs. Model evaluation was based on the accuracy of leave-one-out cross validation (LOOCV) on the training samples. Prediction for a validation cell line is made by the combination of the prediction from the individual models whose LOOCV accuracy >=87.5% (i.e. no or one mistake among the 8 training samples). In detail, a prediction score, Score, for a testing sample is defined as

$$Score = {\sum }_{i = 1}^{5} {a}_{i} {p}_{i} {s}_{i}$$

Where for each classification model i (i=1,2,3,4,5), aᵢ is its LOOCV accuracy, pᵢ is its prediction for the sample with 1 for sensitive and −1 for resistant, sᵢ is a switch between 0 and 1 and is set to 1 when aᵢ>=87.5%; otherwise, 0. The final response prediction for the cell line is sensitive if Score>0 or resistant if Score<0; otherwise, unknown.

### Copy Number and MicroRNA Analysis

DNA copy number (CN) data was generated on Affymetrix Human Mapping 500K Array Set according to the manufacture's protocol (Affymetrix, Santa Clara, Calif.) for 13 cell lines (Table 1). The CN data were imported and analyzed in Partek (http://_www_partek_com, version 6.4) via its copy number workflow. Hidden Markov Model was used to identify copy number variation (CNV) regions between resistant and sensitive cell lines and the significance was assessed by Chi-squared test (p-value threshold was set to 0.01). Mapping genes into the detected CNV regions was done via Affymetrix HG-U133_Plus 2 annotation file.

The microRNA expression profiling was obtained from the Sanger Cell Line Project, under the Cancer Program Data Sets collected at Broad Institute (http://_www_broadinstitute_org/_cgi-bin/_cancer/_datasets_cgi). The collection had 18 NSCLC cell lines that overlapped with what we had (Table 1). ArrayStudio was used to conduct the analysis.

### Results

### Lung Cancer Training Set Cell Lines

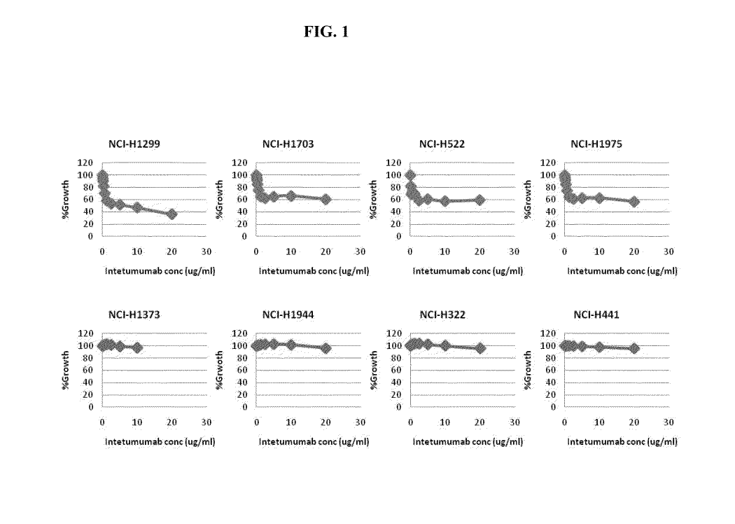

Lung cancer cells were incubated on top of vitronectin—coated plates and assayed for their proliferation/viability in response to increasing concentration of Intetumumab (FIG. 1). A cell line was designed “sensitive” when the cell proliferation index (% growth compared to non-treated control) was at or below 80% at Intetumumab concentration of 20 μg/ml. The cell line was designed “resistant” when the cell proliferation index was above 80%. Sensitive cell lines (NCI-H1299, NCI-H1703, NCI-H522 and NCI-H1975) had proliferation index ranging from 38.1% to 63.3% of the control in response to Intetumumab. Resistant cell lines (NCI-H1373, NCI-H1944, NCI-H322 and NCI-H441) had proliferation index ranging from 95.4% to 96.9% of the control in response to Intetumumab.

### Differentially Expressed Genes in the Training Set Concentrated on Several Chromosomal Locations

Feature filtering approach I/NI used to evaluate differentially expressed genes between sensitive and resistant cell lines yielded a 29,298 probe set (53.6% of total). Largely overlapping probe set was obtained using the USE-Fold approach independently from I/NI. Noticeably, the more stringent the confidence level was chosen (i.e. smaller p-value), the larger overlapping between the selected features of I/NI and USE-Fold was observed. For example, when p=0.0001, 99.8% of the signals selected by USE-Fold also passed I/NI filtering, indicating that the two gene selection algorithms are highly consistent with each other.

Further requirement for an at least 2-fold expression change between sensitive and resistant cell lines reduced the number of the selected probesets to 2919 with 1561 up-regulated and 1358 down-regulated in the resistant cell lines. Details of the number of the selected probe sets under different methods and parameters are shown in Table 6. Analysis on the selected probesets showed their strong enrichment in several chromosomal locations. For example, the 1358 down-regulated probesets in the resistant cell lines are highly enriched on Chromosome (Chr) 19p (hypergeometric test p=0.0001, specifically, 19p12 (p<0.0001) and 19p13 (p<0.0001) regions), 6q (p=0.0017) and 7p (p=0.003) while up-regulated genes reside on 4q (p<0.0001), 1q (p=0.0007) and 8q (p=0.0017). Similar characters were also observed from the genes selected under different parameters (data not shown).

TABLE 6

| | USE-Fold Confidence Level p-value | | | |
|---|---|---|---|---|
| | 0.05 | 0.01 | 0.001 | 0.0001 |
| USE-Fold* | 18616 | 13568 | 9215 | 6430 |
| USE-Fold and I/NI** | 17399 (93.5%) | 13293 (98.0%) | 9161 (99.4%) | 6419 (99.8%) |
| USE-Fold and I/NI and 2-Fold*** | 3713 | 3601 | 3328 | 2919 |
| | (1897 + 1816) | (1838 + 1763) | (1723 + 1605) | (1561 + 1358) |

*Number of probe sets only from USE-Fold
**Number of probe sets selected by both USE-Fold and I/NI with number in parenthesis indicating percentage of this number over the corresponding number in the above row.
***Number of probe sets in “USE-Fold an I/NI” with at least 2-fold change. In parenthesis is shown number of upregulated/downregulated prob sets in the resistant cell lines

### Developing Sensitivity Prediction Markers

Several approaches to select prediction markers were evaluated based on initial results as described above.

In the first approach, the 10 most significantly differentially expressed probes from the 2919 probe set based on I/IN and USE-Fold in addition to the at least 2-fold differential regulation were studied (“Set 1”) (Table 7). The five classification/prediction models used are described above. All the five models achieved 87.5% LOOCV accuracy on training samples.

In the second approach, the top 10 probe sets with largest fold change, including five upregulated and dfive downregulated genes in the resistant vs. sensitive cell liens were selected. (“Set 2”) (Table 8). Four out of the five classification/prediction models achieved 87.5% accuracy on LOOCV in the training set.

In the third approach, the top 10 most significantly differentially regulated genes (t-test) were selected that reside on Chr19p12-13 (“Set 3”) (Table 9). With this gene set, all classification/prediction models achieved >=87.5% accuracy on LOOCV in the training set.

TABLE 7

| Probe Set ID | Gene Symbol | p-vlaue |
|---|---|---|
| 224463_s_at | C11orf70 | 5.31E−08 |
| 241198_s_at | C11orf70 | 1.71E−06 |
| 230747_s_at | TTC39C | 1.12E−05 |
| 218147_s_at | GLT8D1 | 1.72E−05 |
| 205780_at | BIK | 1.97E−05 |
| 223805_at | OSBPL6 | 8.74E−05 |
| 238856_s_at | PANK2 | 8.91E−05 |
| 232202_at | — | 0.0001 |
| 204678_s_at | KCNK1 | 0.0002 |
| 239217_x_at | ABCC3 | 0.0002 |

TABLE 8

| | | | Direction |
|---|---|---|---|
| Probe Set ID | Gene Symbol | p-value | (resistant/sensitive) |
| 201387_s_at | UCHL1 | 0.009 | UP |
| 1567912_s_at | CT45-4 | 0.0345 | UP |
| 225710_at | GNB4 | 0.0419 | UP |
| 206858_s_at | HOXC4 /// HOXC6 | 0.0051 | UP |
| 209118_s_at | TUBA1A | 0.0136 | UP |
| 231736_x_at | MGST1 | 0.0374 | Down |
| 1565162_s_at | MGST1 | 0.0354 | Down |
| 33323_r_at | SFN | 0.0174 | Down |
| 201131_s_at | CDH1 | 0.0193 | Down |
| 224650_at | MAL2 | 0.0074 | Down |

TABLE 9

| Probe Set ID | Gene Symbol | p-value | Chromosomal Location |
|---|---|---|---|
| 203718_at | PNPLA6 | 0.0008 | chr19p13.3-p13.2 |
| 37986_at | EPOR | 0.0014 | chr19p13.3-p13.2 |
| 209963_s_at | EPOR | 0.0015 | chr19p13.3-p13.2 |
| 209962_at | EPOR | 0.0016 | chr19p13.3-p13.2 |
| 242915_at | ZNF682 | 0.0044 | chr19p12 |
| 244552_at | ZNF788 | 0.005 | chr19p13.2 |
| 202927_at | PIN1 | 0.0064 | chr19p13 |
| 223024_at | AP1M1 | 0.0067 | chr19p13.12 |
| 212512_s_at | CARM1 | 0.009 | chr19p13.2 |
| 223318_s_at | ALKBH7 | 0.0091 | chr19p13.3 |

### Predicting Sensitivity Based on Selected Models in the Validation Set

Additional 15 NSCLC cell lines (validation set) were used to validate sensitivity and resistance marker sets as described above to intetumumab.

Using “Set 1”, 8 lung cancer cell lines in the testing set were predicted as sensitive and 7 were predicted to be resistant. Using “Set 2”, 7 cell lines were predicted as sensitive and 8 as resistant. Using “Set 3”, 5 cell lines were predicted as sensitive while 10 were predicted resistant.

To validate the treatment response signatures, in vitro proliferation assay were conducted on the 15 testing cell lines. The predictions using “Set 3” genes was 100% accurate when compared to the in vitro proliferation results (Table 10).

Copy Number Variation (CNV) Overlay with Differential Gene Expression in Resistant and Sensitive Cell Lines

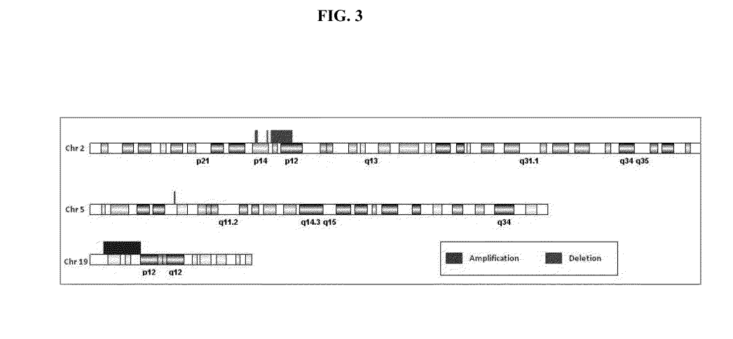

CNV analysis was done for 13 lung cancer cell lines (8 resistant and 5 sensitive) these cell lines are those listed in Table 5 with “Y” under Column “Copy Number Data Available”. Total of 60 significant CNV regions were detected between resistant and sensitive cell lines. Among these regions, 13 of them were amplified while 8 of them were deleted in at least 7 resistant and no more than one sensitive cell line (FIG. 3). Interestingly,

TABLE 10

| | In vitro | | | |
|---|---|---|---|---|
| Cell Line | Response | “Set 1” | “Set 2” | “Set 3” |
| NCI-H1155 | Sensitive | Resistant | Sensitive | Sensitive |
| NCI-H1581 | Sensitive | Sensitive | Sensitive | Sensitive |
| NCI-H2106 | Sensitive | Sensitive | Sensitive | Sensitive |
| NCI-H226 | Sensitive | Sensitive | Sensitive | Sensitive |
| NCI-H510A | Sensitive | Sensitive | Resistant | Sensitive |
| A549 | Resistant | Sensitive | Sensitive | Resistant |
| NCI-H1355 | Resistant | Sensitive | Resistant | Resistant |
| NCI-H1395 | Resistant | Resistant | Resistant | Resistant |
| NCI-H1650 | Resistant | Resistant | Resistant | Resistant |
| NCI-H2122 | Resistant | Resistant | Resistant | Resistant |
| NCI-H2126 | Resistant | Resistant | Resistant | Resistant |
| NCI-H2170 | Resistant | Resistant | Resistant | Resistant |
| NCI-H23 | Resistant | Sensitive | Sensitive | Resistant |
| NCI-H358 | Resistant | Resistant | Resistant | Resistant |
| NCI-H460 | Resistant | Sensitive | Sensitive | Resistant |

12/13 amplified regions are located on 2p12-14 and all 8 deleted regions are located on 19p12-13. 69 genes mapped into the 13 amplified regions and 382 genes within the 8 deleted regions. From these genes, 18 were differentially expressed between resistant and sensitive cell lines. 15 of these were down-regulated and located on 19p, and 3 of these were up-regulated and located on 2p. The genes are shown in Table 11. Among the 15 common genes on 19p, 9 locate at 19p13.2, including lung cancer tumor suppressor gene SMARCA4 (Medina, 2008; Rodriguez, 2009), 3 on 19p13.11, and 3 on 19p12.

A classification model was built using these 18 genes (“Set 4”) and yielded very good LOOCV accuracy on all 23 cell lines—the overall accuracy was 95.7% with only one sensitive cell line being wrongly predicted as resistant.

TABLE 11

| Gene | | Chromosomal | Expres- |
|---|---|---|---|
| Symbol | Gene name | location | sion* |
| MARCH2 | membrane-associated ring | 19p13.2 | Down |
| | finger (C3HC4) 2 | | |
| CACNA1A | calcium channel, voltage- | 19p13.2-13.1 | Down |
| | dependent, P/Q type, alpha | | |
| | 1A subunit | | |
| ZNF44 | Zinc finger protein 44 | 19p13.2 | Down |
| SMARCA4 | SWI/SNF related, matrix | 19p13.2 | Down |
| | associated, actin dependent | | |
| | regulator of chromatin, subf | | |
| LOC147727 | hypothetical LOC147727 | 19p13.2 | Down |
| ZNF823 | zinc finger protein 823 | 19p13.2 | Down |
| ZNF266 | zinc finger protein 266 | 19p13.2 | Down |
| ZNF788 | zinc finger family member 788 | 19p13.2 | Down |
| ZNF709 | zinc finger protein 709 | 19p13.2 | Down |
| C19orf42 | chromosome 19 open reading | 19p13.11 | Down |
| | frame 42 | | |
| ISYNA1 | myo-inositol 1-phosphate | 19p13.11 | Down |
| | synthase A1 | | |
| ZNF14 | zinc finger protein 14 | 19p13.11 | Down |
| ZNF93 | zinc finger protein 93 | 19p12 | Down |
| ZNF253 | zinc finger protein 253 | 19p12 | Down |
| ZNF682 | zinc finger protein 682 | 19p12 | Down |
| EFEMP1 | EGF-containing fibulin-like | 2p16.1 | Up |
| | extracellular matrix protein 1 | | |
| CYP26B1 | cytochrome P450, family 26, | 2p13.3 | Up |
| | subfamily B, polypeptide 1 | | |
| FAM176A | family with sequence similarity | 2p12 | Up |
| | 176, member A | | |

*Differential expression in resistant vs. sensitive cell line

### MicroRNA Profiling Revealed a Signature of Epithelial to Mesenchymal Transition (EMT) and Metastasis

MicroRNA (miRNA) expression data for 18 cell lines, 11 resistant and 7 sensitive were obtained from public domain. These cell lines are those listed in Table 5 with “Y” under Column “MicroRNA Data Available. Since there were multiple screens of these cell lines, the total number of samples included 33 resistant and 16 sensitive ones. With false discovery rate (FDR) set at 0.05, a set of miRNAs were identified that separates resistant and sensitive cell lines (Table 12). The classification model built on this set of miRNAs achieved 95.9% overall accuracy on LOOCV with misclassification on only one resistant and one sensitive samples (97.0% sensitivity and 93.8% specificity).

TABLE 12

| | Fold Change | | |
|---|---|---|---|
| microRNA | (Resistant vs. sensitive) | P-Value | FDR* |
| hsa-miR-335 | 10.44 | 3.84E−08 | 2.23E−05 |
| hsa-miR-141 | 17.51 | 0.0002 | 0.019 |
| hsa-miR-205 | 5.31 | 0.0003 | 0.02 |
| hsa-miR-200c | 17.22 | 0.0005 | 0.0239 |
| hsa-miR-200b | 14.31 | 0.0009 | 0.0391 |
| hsa-miR-130a | −11.46 | 2.61E−05 | 0.0051 |
| hsa-miR-10b | −9.97 | 1.09E−06 | 0.0003 |
| hsa-miR-218 | −3.2 | 0.0003 | 0.02 |

*False Discovery Rate

The microRNAs with higher expression level in the resistant cell lines are miR-335, miR-205 and three members of miR-200 family (miR-141/200b/200c). Interestingly, most of these miRNAs regulate two common processes—epithelial to mesenchymal transition (EMT) and tumor metastasis. The miR-200 family and miR-205 have been previously reported to regulate EMT by targeting ZEB 1 (zinc finger E-box binding homeobox 1) and ZEB2 (zinc finger E-box binding homeobox 2). Furthermore, a recent study found that expression of miR-200 family regulates lung tumor cell metastasis by responding to contextual extracellular signals. On the other hand, miR-130a, a microRNA with the most reduced expression in the resistant cell lines has been reported to regulate angiogenesis by down-regulating two antiangiogenic genes GAX and HOXA5. In addition, miR-10b, which is also down-regulated in the resistant cell lines, is an indication marker of lung metastasis and a direct target of TWIST, a gene which can enhance tumor invasion and metastasis. All these EMT related genes are differentially expressed between resistant and sensitive cell lines.

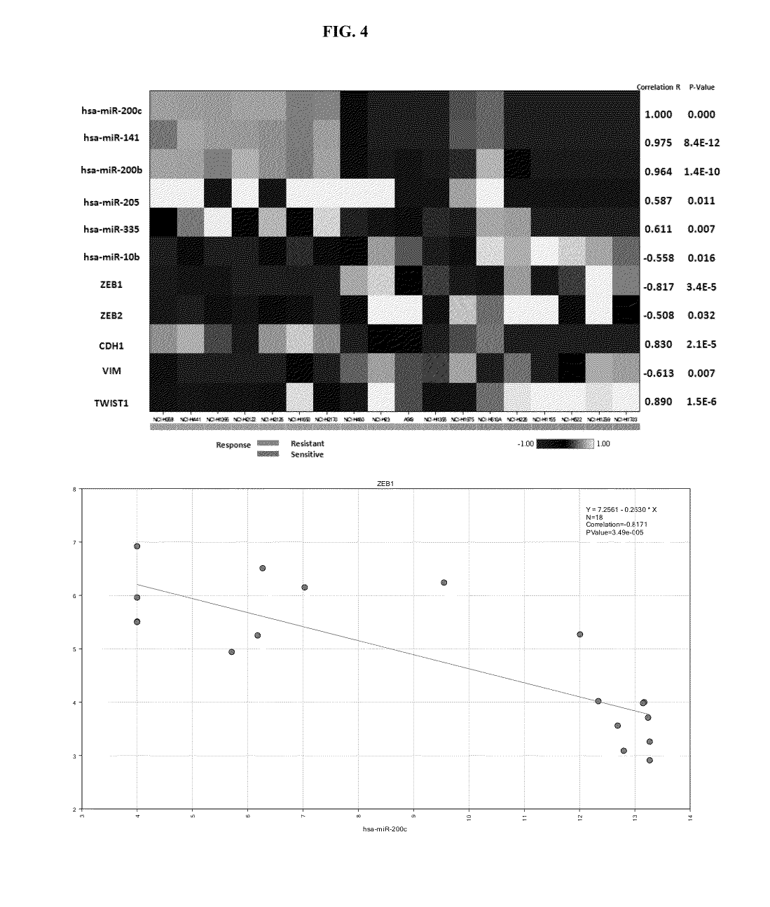

To assess the correlation among miRNA regulators, their targeted genes and between these two groups, we built up a heat map of their expression levels (FIG. 4(A)) and calculated for each of them the Pearson's correlation coefficient to miR-200c (FIG. 4 (A) right). The calculation shows a positive correlation between miR-200c and miR-141, miR-200b, miR-205 and miR-335, and an anti-correlation between miR-200c and miR-10b. Gene wise, ZEB1, ZEB2 and VIM shows significant positive correlation to miR-200C and CDH1 shows negative correlation. Furthermore, TWIST1 and miR-10b also had a strong positive correlation implying their regulatory relationship. FIG. 4(B) further illustrates the strong anti-correlation of miR-200c and its targets ZEB 1 and ZEB2.

### Discussion

This study demonstrated an integrated use of gene expression and DNA copy number variation profiles to predict intetumumab sensitivity of human lung cancer cell lines. The distribution of the identified genes pointed out that several chromosomal locations may be related to the drug sensitivity. Further analysis of DNA copy number data also confirmed deletions on Chr19p in the resistant cell lines. Models built on genes from only the deleted regions yielded very precise predictions on drug response.

One of the noteworthy genes in the deleted chromosome 19p13 region is SMARCA4, a SWI/SNF related, matrix associated, actin dependent regulator of chromatin, also called as BRG1. Known as a tumor suppressor in lung cancer, SMARCA4, along with ZEB1, is known as a new transcriptional mechanism regulating E-cadherin expression and epithelial-to-mesenchymal transdifferentiation that may be involved during the initial stages of tumor invasion. Our results showed that ZEB1 expression was upregulated in resistant cells in which the E-cadherin expression is down-regulated. But, in these resistant cell lines, SMARCA4 region was shown to be deleted. This suggests that there will be SMARCA4-independent mechanism(s) for ZEB1 to repress E-cadherin expression.

Other well-known tumor suppressor gene in this locus (chromosome 19p13.3) is STK11, also known as LKB1. This gene, which encodes a member of the serine/threonine kinase family, regulates cell polarity and functions as a tumor suppressor. STK11 is shown to be mutated in 30% of NSCLC tumors, and recent evidence points to a prominent role in NSCLC metastasis through lysyl oxidase and extracellular matrix remodeling. Interestingly, most of the lung cell lines with deleted or mutated STK11 were found to be resistant in our cell viability/proliferation assay. STK11 status with the addition of K-RAS mutation status would be a useful prognostic marker for Intetumumab resistance.

Moreover, independent from gene expression data, we also obtained a panel of microRNA signatures which showed large difference on their expressions from sensitive to resistant cell lines. Remarkably, most of these microRNAs, that played roles in EMT and tumor metastasis, showed a tight correlation with the known EMT markers that were also found from our differentially expressed gene list.

Although the loss of heterozygosity on Chr19p has been observed in ˜80% of lung tumors (34), it has different distributions between primary and metastatic cancers. In a study conducted by Goeze et al, Chromosomal imbalances of primary and metastatic lung adenocarcinomas, J. Pathol., 2002, 196(1): p. 8-16, losses on Chr19, gains on Chr4q and several other chromosomal locations were reported to be prevalent in non-metastasizing tumors. Therefore, our finding of Chr19p deletion in resistant cell lines is highly consistent with the indication from our microRNA signature, supporting the hypothesis that Intetumumab sensitive cell lines were under-going metastasis.

In summary, our work successfully identified independent gene and microRNA signatures for in vitro response to Intetumumab, an anti-integrin monoclonal antibody. This in vitro study guarantees further in vivo pharmacology studies on Intetumumab. These signatures will eventually guide us to understand the Intetumumab activity in the tumor microenvironment and metastasis. As well, it will directly impact the future drug discovery and development effort on anti-metastasis treatment and patient stratification strategy.

1. A method of identifying a subject with cancer who is most likely to benefit from treatment with anti-integrin antibody Intetumumab, comprising
a) obtaining a sample of nucleic acids from a specimen obtained from a subject with cancer;
b) determining expression levels of nucleic acids hybridizing with a first panel of probes having sequences of SEQ ID NOs: 1-10 or fragments thereof;
c) calculating a prediction score (Score) for the first panel of probes, wherein the prediction score is defined as
$$Score = {\sum }_{i = 1}^{5} {a}_{i} {p}_{i} {s}_{i}$$
where for each classification model i (i=1,2,3,4,5), aᵢ is its leave-one-out cross validation (LOOCV) accuracy, pᵢ is its prediction for the sample with 1 for sensitive and −1 for resistant, sᵢ is a switch between 0 and 1 and is set to 1 when aᵢ>=87.5%; otherwise, 0; and
d) identifying the subject as one most likely to benefit from treatment with the anti-integrin antibody Intetumumab when the calculated prediction score is over zero (>0).

2. A method identifying a subject with cancer who is most likely to benefit from treatment with anti-integrin antibody Intetumumab, comprising
a) obtaining a sample of nucleic acids from a specimen obtained from a subject with cancer;
b) determining expression levels of nucleic acids hybridizing with a first panel of probes having sequences of SEQ ID NOs: 19-28 or fragments thereof;
c) calculating a prediction score (Score) for the first panel of probes, wherein the prediction score is defined as
$$Score = {\sum }_{i = 1}^{5} {a}_{i} {p}_{i} {s}_{i}$$
where for each classification model i (i=1,2,3,4,5), aᵢ is its leave-one-out cross validation (LOOCV) accuracy, pᵢ is its prediction for the sample with 1 for sensitive and −1 for resistant, sᵢ is a switch between 0 and 1 and is set to 1 when aᵢ>=87.5%; otherwise, 0; and
d) identifying the subject as one most likely to benefit from treatment with the anti-integrin antibody Intetumumab when the calculated prediction score is over zero (>0).

3. A method identifying a subject with cancer who is most likely to benefit from treatment with anti-integrin antibody Intetumumab, comprising
a) obtaining a sample of nucleic acids from a specimen obtained from a subject with cancer;
b) determining expression levels of nucleic acids hybridizing with a first panel of probes having sequences of SEQ ID NOs: 39-48 or fragments thereof;
c) calculating a prediction score (Score) for the first panel of probes, wherein the prediction score is defined as
$$Score = {\sum }_{i = 1}^{5} {a}_{i} {p}_{i} {s}_{i}$$
where for each classification model i (i=1,2,3,4,5), aᵢ is its leave-one-out cross validation (LOOCV) accuracy, pᵢ is its prediction for the sample with 1 for sensitive and −1 for resistant, sᵢ is a switch between 0 and 1 and is set to 1 when aᵢ>=87.5%; otherwise, 0; and
d) identifying the subject as one most likely to benefit from treatment with the anti-integrin antibody Intetumumab when the calculated prediction score is over zero (>0).

4. A method identifying a subject with cancer who is most likely to benefit from treatment with anti-integrin antibody Intetumumab, comprising
a) obtaining a sample of nucleic acids from a specimen obtained from a subject with cancer;
b) determining expression levels of nucleic acids hybridizing with a first panel of probes having sequences of SEQ ID NOs: 52, 57 70 or fragments thereof;
c) calculating a prediction score (Score) for the first panel of probes, wherein the prediction score is defined as
$$Score = {\sum }_{i = 1}^{5} {a}_{i} {p}_{i} {s}_{i}$$
where for each classification model i (i=1,2,3,4,5), aᵢ is its leave-one-out cross validation (LOOCV) accuracy, pᵢ is its prediction for the sample with 1 for sensitive and −1 for resistant, sᵢ is a switch between 0 and 1 and is set to 1 when aᵢ>=87.5%; otherwise, 0; and
d) identifying the subject as one most likely to benefit from treatment with the anti-integrin antibody Intetumumab when the calculated prediction score is over zero (>0).
